(19) United States
(12) Patent Application Publication
(10) Pub. No.: US 2024/0050344 A1
(43) Pub. Date: Feb. 15, 2024

(54) Methods of Enhancing Natural Defense in the Oral Cavity

(71) Applicant: Colgate-Palmolive Company, New York, NY (US)

(72) Inventors: Ying YANG, Monmouth Junction, NJ (US); Shaopeng XU, Guangzhou (CN); Guofeng XU, Plainsboro, NJ (US); Harsh Mahendra TRIVEDI, Hillsborough, NJ (US); James MASTERS, Ringoes, NJ (US); Dandan CHEN, Bridgewater, NJ (US); Betty WON, Princeton Junction, NJ (US)

(73) Assignee: Colgate-Palmolive Company, New York, NY (US)

(21) Appl. No.: 18/549,085

(22) PCT Filed: Mar. 11, 2021
(86) PCT No.: PCT/CN2021/080127

§ 371 Date: Sep. 5, 2023

(51) Int. Cl. A61K 8/44; A61K 8/27; A61Q 11/00; A61K 8/21

(52) U.S. Cl. CPC A61K 8/44; A61K 8/27; A61Q 11/00; A61K 8/21; A61K 2800/92

(57) ABSTRACT

Methods of increasing sIgA levels in an individual's oral cavity are disclosed. The methods comprise applying effective amounts of zinc oxide, zinc citrate, and arginine Methods of increasing the concentration of sIgA in the individual's mucosa pellicle in the oral cavity are disclosed. Methods of increasing phagocytotic activity of phagocytotic cells in the individual's oral cavity are also disclosed. In addition, methods of increasing levels of β-Defensin 1 in the individual's oral cavity are disclosed.

### BACKGROUND

A natural human defense system constantly monitors the bacterial colonization and prevents bacterial invasion of local tissues. Mucosa, which lines body cavities that are exposed to the external environment, is the body's first line of defense to protect tissue/body from potentially harmful invaders such as bacteria, viruses, and irritants that can pose threats to good health.

The mouth harbors a diverse, abundant and complex microbial community. Oral mucosa covers about 80% of oral cavity surface and plays an important role maintaining oral health. In the mouth, the natural human defense system includes antibodies, proteins and antimicrobial peptides and cells of the immune system.

The mucosa pellicle is a film structure which coats oral epithelial surface and functions as a moisture retainer, protective barrier and lubricant. Formation of the oral mucosa pellicle is an active and selective process which involves in saliva component and epithelial cell interaction. The mucosa pellicle includes protective salivary proteins such as mucins, Immunoglobulin A (IgA), which are a type of antibody, and β-Defensins.

Secretory IgA (sIgA) is a subclass of IgA, play a critical role in mucosal immunity as part of the first immune defense for the mucosal tissues against the pathogens. SIgA antibodies are the main immunoglobulin found in mucous secretions from, inter alia, salivary glands. SIgA antibodies are not synthesized by mucosal epithelial cells; they are produced by B-lymphocytes adjacent to the mucosal cells, then transported through the cell interiors, and released into the secretions from the cells. Part of the natural defense system, salivary sIgA act as a specific host defense factors in saliva. On the oral mucosal surface, sIgA binds with mucin to form a protecting complex in the bound mucosa pellicle. The concentration of sIgA on oral surfaces prevents microorganisms binding to mucosal surface.

β-Defensins are cationic peptides with broad-spectrum antimicrobial activity that are produced by epithelia at mucosal surfaces and play an important in the innate defenses against oral microorganisms.

Immune-surveillance and antimicrobial functions of polymorphonuclear leukocytes (PMNs), which migrate from the circulatory system through the oral mucosal tissues as oral PMNs (oPMNs), also play an important role in protection against foreign invaders. PMNs include neutrophils, which are a type of phagocyte, are a part of the first line of defense against potential pathogens. The oral cavity contains macrophages, which are also phagocytes. Phagocytosis is a very important physiological process which is characterized by the ingestion of foreign particles and killing of microorganisms by phagocytic leukocytes.

Strengthening human natural defenses reduces the risk of infection and therefore can play a role in a strategy to prevent bacterial-induced oral problems and diseases. Improving human natural defenses promotes good oral health.

### BRIEF SUMMARY

Methods of increasing sIgA levels in an individual's oral cavity are provided. The methods comprise applying twice daily for seven consecutive days to the individual's oral cavity an oral care composition that comprises an effective amount of zinc oxide, zinc citrate, and arginine.

In some embodiments, methods are provided which comprises applying the oral care composition in an amount effective to increase the concentration of sIgA in the individual's mucosa pellicle in the oral cavity.

In some embodiments, methods are provided which comprises applying the oral care composition in an amount effective to increase phagocytotic activity of phagocytotic cells in the individual's oral cavity.

In some embodiments, methods are provided which comprises applying the oral care composition in an amount effective to increase levels of β-Defensin 1 in the individual's oral cavity.

### BRIEF DESCRIPTION OF THE DRAWINGS

FIG. 1 contains data from experiments described in Example 1. Data in FIG. 1 show that application of an oral care composition comprising zinc oxide, zinc citrate, and arginine twice daily for seven consecutive days significantly increased the sIgA levels as a percentage of total mucosal protein on the surface of an individual's oral cavity.

FIG. 2 contains data from experiments described in Example 2. Data in FIG. 2 show that zinc is delivered to the oral surface after brushing with an oral care composition that comprises zinc oxide, zinc citrate, and arginine and that detectable levels of the zinc one hour after brushing.

FIG. 3 contains data from experiments described in Example 2. Data in FIG. 3 show that zinc is not detectable on samples of oral surface cells one hour after brushing with an oral care composition that does not contain zinc oxide, zinc citrate, and arginine.

FIG. 4 contains data from experiments described in Example 2. Data in FIG. 4 show that one hour after brushing with an oral care composition that comprises zinc oxide, zinc citrate, and arginine, sIgA is concentrated on the oral surface.

FIG. 5 contains data from experiments described in Example 2. Data in FIG. 5 show that sIgA is not detectable on samples of oral surface cells one hour after brushing with an oral care composition that does not contain zinc oxide, zinc citrate, and arginine.

FIG. 6 contains data from experiments described in Example 3. Data in FIG. 6 show that ZnCl₂ increases phagocytosis activity of immune cells and that chelation of zinc by EDTA reduces the effect of zinc in doing so.

FIG. 7 contains data from experiments described in Example 3. Data in FIG. 7 show that ZnO increases phagocytosis activity of immune cells and that in some amounts chelation of zinc by EDTA reduces the effect of zinc in doing so.

FIG. 8 contains data from experiments described in Example 4. Data in FIG. 8 show that toothpaste that contains ZnO increases production of β-defensin 1 in oral epithelial tissue.

### DETAILED DESCRIPTION

As disclosed herein, application of an effective amount of an oral care composition that comprise zinc oxide, zinc citrate, and arginine to the oral cavity of an individual daily for seven days promoted increased levels of sIgA in the oral cavity. Application of zinc was also observed to promote immune cell activity, increasing phagocytosis activity of phagocytic immune cells. Moreover, oral epithelial tissue was induced by zinc to increase secretion of the antimicrobial β-Defensins.

Zinc is known to play a role in the human immune system. It is crucial for normal development and function of cells mediating innate immunity, neutrophils and natural killer cells. Zinc deficiency affects cells involved in both innate and adaptive immunity at the survival, proliferation and maturation levels. These cells include monocytes, polymorphonuclear-, natural killer-, T-, and B-cells. While acute Zn deficiency causes a decrease in innate and adaptive immunity, chronic deficiency increases inflammation. Zinc deficient persons experience increased susceptibility to infection.

Despite being known to have a role in a properly functioning immune system, the increased levels of sIgA in the oral cavity following application of an oral composition comprising zinc oxide, zinc citrate, and arginine to the oral cavity for seven days provides surprising enhancement to the natural immune system. Following application of zinc, sIgA was observed to be present at a higher level on the oral epithelial cells surface. Application of zinc resulted in higher concentrations of sIgA in the mucosa pellicle. Application of zinc was also observed to increase phagocytosis activity of phagocytic immune cells. Moreover, oral epithelial tissue was induced by zinc to increase secretion of antimicrobial β-Defensins in oral tissue.

The unexpected increase in sIgA in the oral cavity following application of zinc oxide, zinc citrate, and arginine to the oral cavity for greater than three consecutive days strengthens the human natural defenses, improves protection against pathogenic microbes and promote good oral health. In some embodiments, zinc oxide, zinc citrate, and arginine are applied to the oral cavity for at least four consecutive days, for four or more consecutive days, for at least five consecutive days, for five or more consecutive days, for at least four consecutive days, for four or more consecutive days, for at least five consecutive days, for five or more consecutive days, for at least six consecutive days, for six or more consecutive days, for at least seven consecutive days, or for seven or more consecutive days. In such embodiments, the zinc oxide, zinc citrate, and arginine are applied to the oral cavity at least one time, and more preferable two times per day. In some embodiments, the unexpected increase in sIgA in the oral cavity following application of zinc oxide, zinc citrate, and arginine to the oral cavity two times per day for seven consecutive days strengthens the human natural defenses, improves protection against pathogenic microbes and promote good oral health. The increased concentration of sIgA in the mucosa pellicle, the increase in phagocytic activity of immune cells and the increase secretion of antimicrobial β-Defensins by oral epithelial tissue advantageously further enhance and strengthen natural defenses in the oral cavity.

Application of an oral composition comprising zinc oxide, zinc citrate, and arginine to the oral cavity daily or twice daily for more than three consecutive days can produce increases sIgA levels in the oral cavity. In some embodiments, an oral composition comprising zinc oxide, zinc citrate, and arginine is applied to the oral cavity daily or twice daily for at least four consecutive days, for four or more consecutive days, for at least five consecutive days, for five or more consecutive days, for at least four consecutive days, for four or more consecutive days, for at least five consecutive days, for five or more consecutive days, for at least six consecutive days, for six or more consecutive days, for at least seven consecutive days, or for seven or more consecutive days. Application of an oral composition comprising zinc oxide, zinc citrate, and arginine to the oral cavity daily or more preferably twice daily for seven consecutive days produces increases sIgA levels in the oral cavity. The oral care composition is applied to the oral cavity in an amount effect to promote increased concentration of sIgA. Additional beneficial effects of using an oral composition comprising zinc include increased concentration of sIgA in the mucosa pellicle on the oral epithelial surface, enhanced phagocytic activity of phagocytic immune cells and increased secretion of antimicrobial β-Defensins by oral epithelial tissue.

Application of an oral composition comprising zinc oxide, zinc citrate, and arginine to the oral cavity daily or twice daily for greater than three consecutive days can provide benefits by strengthening the natural defenses and promoting good oral health. In some embodiments, an oral composition comprising zinc oxide, zinc citrate, and arginine is applied to the oral cavity daily or twice daily for at least four consecutive days, for four or more consecutive days, for at least five consecutive days, for five or more consecutive days, for at least four consecutive days, for four or more consecutive days, for at least five consecutive days, for five or more consecutive days, for at least six consecutive days, for six or more consecutive days, for at least seven consecutive days, or for seven or more consecutive days. Application of an oral composition comprising zinc oxide, zinc citrate, and arginine to the oral cavity daily or more preferably twice daily for seven consecutive days provides benefits by strengthening the natural defenses and promoting good oral health. The increase of sIgA after usage of the oral composition daily or twice daily that comprises zinc oxide, zinc citrate and arginine daily or twice daily for greater than three consecutive days, for at least four consecutive days, for four or more consecutive days, for at least five consecutive days, for five or more consecutive days, for at least four consecutive days, for four or more consecutive days, for at least five consecutive days, for five or more consecutive days, for at least six consecutive days, for six or more consecutive days, for at least seven consecutive days, or for seven or more consecutive days can provide a significant oral health benefit of boosting oral defense power. The increase in IgA on oral epithelial cells surface and the higher concentration of IgA in the mucosa pellicle enhance the mucosal protective property, thus producing a health benefit to the whole mouth. Zinc additionally enhances human natural defense through increasing the phagocytic activity of immune cells and promoting secretion of antimicrobial peptide by oral epithelial tissue.

Some embodiments provided herein include methods that comprise applying to the oral cavity of an individual an effective amount of zinc oxide, zinc citrate, and arginine daily or twice daily for greater than three consecutive days, for at least four consecutive days, for four or more consecutive days, for at least five consecutive days, for five or more consecutive days, for at least four consecutive days, for four or more consecutive days, for at least five consecutive days, for five or more consecutive days, for at least six consecutive days, for six or more consecutive days, for at least seven consecutive days, or for seven or more consecutive days can provide a significant oral health benefit of boosting oral defense power. In some embodiments, an effective amount of an oral care composition that comprise zinc oxide, zinc citrate, and arginine is an amount that increases levels of sIgA in the oral cavity of an individual. In some embodiments provided herein include methods that comprise applying to the oral cavity of an individual an effective amount of zinc oxide, zinc citrate, and arginine daily for seven consecutive days. In some embodiments, an effective amount of an oral care composition that comprise zinc oxide, zinc citrate, and arginine is an amount that increases levels of sIgA in the oral cavity of an individual when applied daily for seven consecutive days.

In some embodiments, methods of increasing sIgA levels in an individual's oral cavity are provided. The methods may comprise applying daily or twice daily for greater than three consecutive days, for at least four consecutive days, for four or more consecutive days, for at least five consecutive days, for five or more consecutive days, for at least four consecutive days, for four or more consecutive days, for at least five consecutive days, for five or more consecutive days, for at least six consecutive days, for six or more consecutive days, for at least seven consecutive days, or for seven or more consecutive days an oral care composition that comprises an effective amount of zinc oxide, zinc citrate, and arginine to increase sIgA in the individual's oral cavity. In some preferred embodiments, methods of increasing sIgA levels in an individual's oral cavity are provided that comprise applying twice daily for seven consecutive days, an effective amount of zinc oxide, zinc citrate, and arginine to increase sIgA in the individual's oral cavity. In some embodiments, the oral care composition is a toothpaste. In some such embodiments, the zinc oxide is present in the oral care composition in an amount of from 0.75 to 1.25 wt %, the zinc citrate is present in the oral care composition in an amount of from 0.25 to 1.0 wt %, and the arginine is present in the oral care composition in an amount of from 0.1% to 15%, based on the total weight of the composition, the weight of the basic amino acid being calculated as free form. In some embodiments, the arginine is L-arginine. In some embodiments, the arginine is in free form. In some embodiments, the arginine is in salt form. In some embodiments, the ratio of the amount of zinc oxide (by wt %) to zinc citrate (by wt %) is 2:1, 2.5:1, 3:1, 3.5:1 or 4:1, based on the total weight of the composition. In some embodiments, the ratio of the amount of zinc oxide (by wt %) to zinc citrate (by wt %) is 2:1, based on the total weight of the composition. In some embodiments, the oral care composition further comprises fluoride. In some embodiments, the oral care composition further comprises stannous fluoride. In some methods, the oral care composition comprises an amount of zinc oxide, zinc citrate, and arginine effective to increase the concentration of sIgA in the individual's mucosa pellicle in the oral cavity.

“Oral care composition” refers to a composition that is delivered to the oral surfaces. The composition may be a product which, during the normal course of usage, is not, the purpose of systemic administration of particular therapeutic agents, intentionally swallowed, but is rather retained in the oral cavity for a time sufficient to contact substantially all of the dental surfaces and/or oral tissues for the purposes of oral activity. Examples of the oral composition include, but are not limited to a toothpaste or a dentifrice, a mouthwash or a mouth rinse, powder (e.g., tooth powder), lozenge, mint, cream, strip or gum (e.g., chewing gum) a topical oral gel, and a denture cleanser and the like.

In some embodiments the oral care compositions comprise zinc oxide to zinc citrate in a ratio from 1.5:1 to 4.5:1, 1.5:1 to 4:1, 1.7:1 to 2.3:1, 1.9:1 to 2.1:1, or about 2:1. Also, the corresponding molar ratios based on these weight ratios can be used. In some embodiments, the total concentration of zinc salts in the composition is from 0.2 weight % to 5 weight %, or from 0.5 weight % to 2.5 weight % or from 0.8 weight % to 2 weight %, or about 1.5 weight %, based on the total weight of the composition. In some embodiments, the molar ratio of arginine to total zinc salts is from 0.05:1 to 10:1. In some embodiments, the composition comprises zinc oxide in an amount of from 0.5 weight % to 1.5 weight % and zinc citrate in an amount of from 0.25 weight % to 0.75 weight %, based on the total weight of the composition. In some embodiments, the composition may comprise zinc oxide in an amount of from 0.75 weight % to 1.25 weigh % and zinc citrate in an amount of from 0.4 weight % to 0.6 weight %, based on the total weight of the composition. In some embodiments, the composition comprises zinc oxide in an amount of about 1 weight % and zinc citrate in an amount of about 0.5 weight %, based on the total weight of the composition. In some embodiments, zinc oxide may be present in an amount of from 0.75 to 1.25 wt % (e.g., 1.0 wt. %) the zinc citrate is in an amount of from 0.25 to 1.0 wt % (e.g. 0.25 to 0.75 wt. %, or 0.5 wt. %) and based on the weight of the oral care composition. In some embodiments, the zinc citrate is about 0.5 wt %. In some embodiments, the zinc oxide is about 1.0 wt %.

In some embodiments the ZnO particles may have an average particle size of from 1 to 7 microns. In some embodiments, the ZnO particles have an average particle size of 5 microns or less. In some embodiments, suitable zinc oxide particles for oral care compositions have, for example, a particle size distribution of 3 to 4 microns, or alternatively, a particle size distribution of 5 to 7 microns, alternatively, a particle size distribution of 3 to 5 microns, alternatively, a particle size distribution of 2 to 5 microns, or alternatively, a particle size distribution of 2 to 4 microns. Zinc oxide may have a particle size which is a median particle size. Suitable particles may have, for example, a median particle size of 8 microns or less, alternatively, a median particle size of 3 to 4 microns, alternatively, a median particle size of 5 to 7 microns, alternatively, a median particle size of 3 to 5 microns, alternatively, a median particle size of 2 to 5 microns, or alternatively, a median particle size of 2 to 4 microns. In another aspect, that particle size is an average (mean) particle size. In an embodiment, the mean particle comprises at least 5%, at least 10%, at least 15%, at least 20%, at least 25%, at least 30%, at least 35%, or at least 40% of the total metal oxide particles in an oral care composition of the invention. The particle may be present in an amount of up to 5% by weight, based on the total weight of the oral care composition, for example in an amount of from 0.5 to 5% by weight, preferably of up to 2% by weight, more preferably from 0.5 to 2% by weight, more preferably from 1 to 2% by weight, or in some embodiment from 2.5 to 4.5% by weight, being based on the total weight of the oral care composition. In some embodiments, the source of zinc oxide particles and/or the form they may be incorporated into the oral care composition in is selected from one or more of a powder, a nanoparticle solution or suspension, or encapsulated in a polymer or bead. Zinc oxide particles may be selected to achieve occlusion of dentin particles. Particle size distribution may be measured using a Malvern Particle Size Analyzer, Model Mastersizer 2000 (or comparable model) (Malvern Instruments, Inc., Southborough, Mass.), wherein a helium-neon gas laser beam is projected through a transparent cell which contains silica, such as, for example, silica hydrogel particles suspended in an aqueous solution. Light rays which strike the particles are scattered through angles which are inversely proportional to the particle size. The photodetector arrant measures the quantity of light at several predetermined angles. Electrical signals proportional to the measured light flux values are then processed by a microcomputer system, against a scatter pattern predicted from theoretical particles as defined by the refractive indices of the sample and aqueous dispersant to determine the particle size distribution of the metal oxide. It will be understood that other methods of measuring particle size are known in the art, and based on the disclosure set forth herein, the skilled artisan will understand how to calculate median particle size, mean particle size, and/or particle size distribution of metal oxide particles.

Oral care compositions comprise arginine or a salt thereof. In some embodiments, the arginine is L-arginine or a salt thereof. Suitable salts include salts known in the art to be pharmaceutically acceptable salts are generally considered to be physiologically acceptable in the amounts and concentrations provided. Physiologically acceptable salts include those derived from pharmaceutically acceptable inorganic or organic acids or bases, for example acid addition salts formed by acids which form a physiological acceptable anion, e.g., hydrochloride or bromide salt, and base addition salts formed by bases which form a physiologically acceptable cation, for example those derived from alkali metals such as potassium and sodium or alkaline earth metals such as calcium and magnesium. Physiologically acceptable salts may be obtained using standard procedures known in the art, for example, by reacting a sufficiently basic compound such as an amine with a suitable acid affording a physiologically acceptable anion. In some embodiments, the arginine in partially or wholly in salt form such as arginine phosphate, arginine hydrochloride or arginine bicarbonate. In some embodiments, the arginine is present in an amount corresponding to 0.1% to 15%, e.g., 0.1 wt % to 10 wt %, e.g., 0.1 to 5 wt %, e.g., 0.5 wt % to 3 wt % of the total composition weight, about e.g., 1%, 1.5%, 2%, 3%, 4%, 5%, or 8%, wherein the weight of the arginine is calculated as free form. In some embodiments the arginine is present in an amount corresponding to about 0.5 wt. % to about 20 wt. % of the total composition weight, about 0.5 wt. % to about 10 wt. % of the total composition weight, for example about 1.5 wt. %, about 3.75 wt. %, about 5 wt. %, or about 7.5 wt. % wherein the weight of the arginine is calculated as free form. In some embodiments, the arginine is present in an amount of from 0.5 weight % to 10 weight %, or from 0.5 weight % to 3 weight % or from 1 weight % to 2.85 weight %, or from 1.17 weight % to 2.25 weight %, based or from 1.4 weight % to 1.6 weight %, or from 0.75 weight % to 2.9 weight %, or from 1.3 weight % to 2 weight %, or about 1.5 weight %, based on the total weight of the composition. Typically, the arginine is present in an amount of up to 5% by weight, further optionally from 0.5 to 5% by weight, still further optionally from 2.5 to 4.5% by weight, based on the total weight of the oral care composition. In some embodiments, arginine is present in an amount from 0.1 wt. %-6.0 wt. %. (e.g., about 1.5 wt %) or from about 4.5 wt. %-8.5 wt. % (e.g., 5.0%) or from 3.5 wt. %-9 wt. % or 8.0 wt. %. In some embodiments, the arginine is present in a dentifrice, at for example about 0.5-2 wt. %, e.g., and about 0.8% in the case of a mouthwash.

One or more fluoride ion sources are optionally present in an amount providing a clinically efficacious amount of soluble fluoride ion to the oral care composition. A fluoride ion source is useful, for example, as an anti-caries agent. Any orally acceptable particulated fluoride ion source can be used, including stannous fluoride, sodium fluoride, potassium fluoride, potassium monofluorophosphate, sodium monofluorophosphate, ammonium monofluorophosphate, sodium fluorosilicate, ammonium fluorosilicate, indium fluoride, amine fluoride such as olaflur (N′-octadecyltrimethylendiamine-N,N,N′-tris(2-ethanol)-dihydrofluoride), ammonium fluoride, titanium fluoride, hexafluorosulfate, and combinations thereof. Fluoride where present may be present at levels of, e.g., about 25 to about 25,000 ppm, for example about 50 to about 5000 ppm, about 750 to about 2,000 ppm for a consumer toothpaste (e.g., 1000-1500 ppm, e.g., about 1000 ppm, e.g., about 1450 ppm), product. In some embodiments, fluoride is present from about 100 to about 1000, from about 200 to about 500, or about 250 ppm fluoride ion. 500 to 3000 ppm. In some embodiments, the fluoride source provides fluoride ion in an amount of from 50 to 25,000 ppm (e.g., 750-7000 ppm, e.g., 1000-5500 ppm, e.g., about 500 ppm, 1000 ppm, 1100 ppm, 2800 ppm, 5000 ppm, or 25000 ppm). In some embodiments, the fluoride source is stannous fluoride. In some embodiments, the fluoride source is stannous fluoride which provides fluoride in an amount from 750-7000 ppm (e.g., about 1000 ppm, 1100 ppm, 2800 ppm, 5000 ppm). In some embodiments, the fluoride source is stannous fluoride which provides fluoride in an amount of about 5000 ppm. In some embodiments, the fluoride source is sodium fluoride which provides fluoride in an amount from 750-2000 ppm (e.g., about 1450 ppm). In some embodiments, the fluoride source is selected from sodium fluoride and sodium monofluorophosphate and which provides fluoride in an amount from 1000 ppm-1500 ppm. In some embodiments, the fluoride source is sodium fluoride or sodium monofluorophosphate and which provides fluoride in an amount of about 1450 ppm. In some embodiments, stannous fluoride is the only fluoride source. In some embodiments, the fluoride source is stannous fluoride which provides fluoride in an amount from 750-7000 ppm (e.g., about 1000 ppm, 1100 ppm, 2800 ppm, 5000 ppm). In some embodiments, the fluoride source is stannous fluoride which provides fluoride in an amount of about 5000 ppm. Fluoride ion sources may be added to the compositions at a level of about 0.001 wt. % to about 10 wt. %, e.g., from about 0.003 wt. % to about 5 wt. %, 0.01 wt. % to about 1 wt., or about 0.05 wt. %. In some embodiment, the stannous fluoride is present in an amount of 0.1 wt. % to 2 wt. % (0.1 wt %-0.6 wt. %) of the total composition weight. Fluoride ion sources may be added to the compositions at a level of about 0.001 wt. % to about 10 wt. %, e.g., from about 0.003 wt. % to about 5 wt. %, 0.01 wt. % to about 1 wt., or about 0.05 wt. %. However, it is to be understood that the weights of fluoride salts to provide the appropriate level of fluoride ion will obviously vary based on the weight of the counter ion in the salt, and one of skill in the art may readily determine such amounts. In some embodiment, the fluoride source is a fluoride salt present in an amount of 0.1 wt. % to 2 wt. % (0.1 wt %-0.6 wt. %) of the total composition weight (e.g., sodium fluoride (e.g., about 0.32 wt. %) or sodium monofluorophosphate). e.g., 0.3-0.4%, e.g., ca. 0.32% sodium fluoride

The oral care compositions described herein may also comprise one or more further agents such as those typically selected from the group consisting of: abrasives, an anti-plaque agent, a whitening agent, antibacterial agent, cleaning agent, a flavoring agent, a sweetening agent, adhesion agents, surfactants, foam modulators, pH modifying agents, humectants, mouth-feel agents, colorants, tartar control (anti-calculus) agent, polymers, saliva stimulating agent, nutrient, viscosity modifier, anti-sensitivity agent, antioxidant, and combinations thereof.

In some embodiments, the oral care compositions comprise one or more abrasive particulates such as those useful for example as a polishing agent. Any orally acceptable abrasive can be used, but type, fineness, (particle size) and amount of abrasive should be selected so that tooth enamel is not excessively abraded in normal use of the composition. Examples of abrasive particulates may be used include abrasives such sodium bicarbonate, insoluble phosphates (such as orthophosphates, polymetaphosphates and pyrophosphates including dicalcium orthophosphate dihydrate, calcium pyrophosphate, tricalcium phosphate, calcium polymetaphosphate and insoluble sodium polymetaphosphate), calcium phosphate (e.g., dicalcium phosphate dihydrate), calcium sulfate, natural calcium carbonate (CC), precipitated calcium carbonate (PCC), silica (e.g., hydrated silica or silica gels or in the form of precipitated silica or as admixed with alumina), iron oxide, aluminum oxide, aluminum silicate, calcined alumina, bentonite, other siliceous materials, perlite, plastic particles, e.g., polyethylene, and combinations thereof. The natural calcium carbonate abrasive of is typically a finely ground limestone which may optionally be refined or partially refined to remove impurities. The material preferably has an average particle size of less than 10 microns, e.g., 3-7 microns, e.g., about 5.5 microns. For example, a small particle silica may have an average particle size (D50) of 2.5-4.5 microns. Because natural calcium carbonate may contain a high proportion of relatively large particles of not carefully controlled, which may unacceptably increase the abrasivity, preferably no more than 0.01%, preferably no more than 0.004%) by weight of particles would not pass through a 325 mesh. The material has strong crystal structure, and is thus much harder and more abrasive than precipitated calcium carbonate. The tap density for the natural calcium carbonate is for example between 1 and 1.5 g/cc, e.g., about 1.2 for example about 1.19 g/cc. There are different polymorphs of natural calcium carbonate, e.g., calcite, aragonite and vaterite, calcite being preferred for purposes of this invention. An example of a commercially available product suitable for use in the present invention includes Vicron® 25-11 FG from GMZ. Precipitated calcium carbonate has a different crystal structure from natural calcium carbonate. It is generally more friable and more porous, thus having lower abrasivity and higher water absorption. For use in the present invention, the particles are small, e.g., having an average particle size of 1-5 microns, and e.g., no more than 0.1%, preferably no more than 0.05% by weight of particles which would not pass through a 325 mesh. The particles may for example have a D50 of 3-6 microns, for example 3.8-4.9, e.g., about 4.3; a D50 of 1-4 microns, e.g., 2.2-2.6 microns, e.g., about 2.4 microns, and a D10 of 1-2 microns, e.g., 1.2-1.4, e.g., about 1.3 microns. The particles have relatively high water absorption, e.g., at least 25 g/100 g, e.g., 30-70 g/100 g. Examples of commercially available products suitable for use include, for example, Carbolag® 15 Plus from Lagos Industria Quimica. In some embodiments, additional calcium-containing abrasives, for example calcium phosphate abrasive, e.g., tricalcium phosphate, hydroxyapatite or dicalcium phosphate dihydrate or calcium pyrophosphate, and/or silica abrasives, sodium metaphosphate, potassium metaphosphate, aluminum silicate, calcined alumina, bentonite or other siliceous materials, or combinations thereof are used. Examples of silica abrasives include, but are not limited to, precipitated or hydrated silicas having a mean particle size of up to about 20 microns (such as Zeodent 105 and Zeodent 1 14 marketed by J.M. Huber Chemicals Division, Havre de Grace, Md. 21078); Sylodent 783 (marketed by Davison Chemical Division of W.R. Grace & Company); or Sorbosil AC 43 (from PQ Corporation). In some embodiments, an effective amount of a silica abrasive is about 10-30%, e.g., about 20%. In some embodiments, the acidic silica abrasive Sylodent is included at a concentration of about 2 to about 35% by weight; about 3 to about 20% by weight, about 3 to about 15% by weight, about 10 to about 15% by weight. For example, the acidic silica abrasive may be present in an amount selected from 2 wt. %, 3 wt. %, 4% wt. %, 5 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %. Sylodent 783 has a pH of 3.4-4.2 when measured as a 5% by weight slurry in water and silica material has an average particle size of less than 10 microns, e.g., 3-7 microns, e.g., about 5.5 microns. In some embodiments, the silica is synthetic amorphous silica, (e.g., 1%-28% by wt.) (e.g., 8%-25% by wt). In some embodiments, the silica abrasives are silica gels or precipitated amorphous silicas, e.g., silicas having an average particle size ranging from 2.5 microns to 12 microns. Some embodiments further comprise a small particle silica having a median particle size (d50) of 1-5 microns (e.g., 3-4 microns) (e.g., about 5 wt. % Sorbosil AC43 from PQ Corporation Warrington, United Kingdom). The composition may contain from 5 to 20 wt % small particle silica, or for example 10-15 wt %, or for example 5 wt %, 10 wt %, 15 wt % or 20 wt % small particle silica. In some embodiments, 20-30 wt % of the total silica in the composition is small particle silica (e.g., having a median particle size (d50) of 3-4 microns and wherein the small particle silica is about 5 wt. % of the oral care composition. In some embodiments, silica is used as a thickening agent, e.g., particle silica. In some embodiments, the composition comprises calcium carbonate, such as precipitated calcium carbonate high absorption (e.g., 20% to 30% by weight of the composition or, 25% precipitated calcium carbonate high absorption), or precipitated calcium carbonate—light (e.g., about 10% precipitated calcium carbonate—light) or about 10% natural calcium carbonate.

In some embodiments, the oral care compositions comprise a whitening agent, e.g., a selected from the group consisting of peroxides, metal chlorites, perborates, percarbonates, peroxyacids, hypochlorites, hydroxyapatite, and combinations thereof. Oral care compositions may comprise hydrogen peroxide or a hydrogen peroxide source, e.g., urea peroxide or a peroxide salt or complex (e.g., such as peroxyphosphate, peroxycarbonate, perborate, peroxysilicate, or persulphate salts; for example, calcium peroxyphosphate, sodium perborate, sodium carbonate peroxide, sodium peroxyphosphate, and potassium persulfate or hydrogen peroxide polymer complexes such as hydrogen peroxide-polyvinyl pyrrolidone polymer complexes.

In some embodiments, the oral care compositions comprise an effective amount of one or more antibacterial agents, for example comprising an antibacterial agent selected from halogenated diphenyl ether (e.g. triclosan), triclosan monophosphate, herbal extracts and essential oils (e.g., rosemary extract, tea extract, magnolia extract, thymol, menthol, eucalyptol, geraniol, carvacrol, citral, hinokitol, magonol, ursolic acid, ursic acid, morin, catechol, methyl salicylate, epigallocatechin gallate, epigallocatechin, gallic acid, miswak extract, sea-buckthorn extract), bisguanide antiseptics (e.g., chlorhexidine, alexidine or octenidine), quaternary ammonium compounds (e.g., cetylpyridinium chloride (CPC), benzalkonium chloride, tetradecylpyridinium chloride (TPC), N-tetradecyl-4-ethylpyridinium chloride (TDEPC)), phenolic antiseptics, hexetidine furanones, bacteriocins, ethyllauroyl arginate, arginine bicarbonate, a Camellia extract, a flavonoid, a flavan, halogenated diphenyl ether, creatine, sanguinarine, povidone iodine, delmopinol, salifluor, metal ions (e.g., zinc salts, stannous salts, copper salts, iron salts), propolis and oxygenating agents (e.g., hydrogen peroxide, buffered sodium peroxyborate or peroxycarbonate), phthalic acid and its salts, monoperthalic acid and its salts and esters, ascorbyl stearate, oleoyl sarcosine, alkyl sulfate, dioctyl sulfosuccinate, salicylanilide, domiphen bromide, delmopinol, octapinol and other piperidino derivatives, nisin preparations, chlorite salts; parabens such as methylparaben or propylparaben and mixtures of any of the foregoing. One or more additional antibacterial or preservative agents may optionally be present in the composition in a total amount of from about 0.01 wt. % to about 0.5 wt. %, optionally about 0.05 wt. % to about 0.1 wt. % or about 0.3%. by total weight of the composition.

In some embodiments, the oral care compositions may comprise at least one bicarbonate salt useful for example to impart a “clean feel” to teeth and gums due to effervescence and release of carbon dioxide. Any orally acceptable bicarbonate can be used, including without limitation, alkali metal bicarbonates such as sodium and potassium bicarbonates, ammonium bicarbonate and the like. The one or more additional bicarbonate salts are optionally present in a total amount of about 0.1 wt. % to about 50 wt. %, for example about 1 wt. % to 20 wt. %, by total weight of the composition.

In some embodiments, the oral care compositions also comprise at least one flavorant, useful for example to enhance taste of the composition. Any orally acceptable natural or synthetic flavorant can be used, including without limitation essential oils and various flavoring aldehydes, esters, alcohols, and similar materials, tea flavors, vanillin, sage, marjoram, parsley oil, spearmint oil, cinnamon oil, oil of wintergreen, peppermint oil, clove oil, bay oil, anise oil, eucalyptus oil, citrus oils, fruit oils, sassafras and essences including those derived from lemon, orange, lime, grapefruit, apricot, banana, grape, apple, strawberry, cherry, pineapple, etc., bean- and nut-derived flavors such as coffee, cocoa, cola, peanut, almond, etc., adsorbed and encapsulated flavorants and the like. Also encompassed within flavorants herein are ingredients that provide fragrance and/or other sensory effect in the mouth, including cooling or wanning effects. Such ingredients illustratively include menthol, carvone, menthyl acetate, menthyl lactate, camphor, eucalyptus oil, eucalyptol, anethole, eugenol, cassia, oxanone, a-irisone, propenyl guaiethoi, thymol, linalool, benzaldehyde, cinnamaldehyde, N-ethyl-p-menthan-3-carboxamine, N,2,3-trimethyl-2-isopropylbutanamide, 3-(1-menthoxy)-propane-1,2-diol, cinnamaldehyde glycerol acetal (CGA), menthone glycerol acetal (MGA) and the like. One or more flavorants are optionally present in a total amount of from about 0.01 wt. % to about 5 wt. %, for example, from about 0.03 wt. % to about 2.5 wt. %, optionally about 0.05 wt. % to about 1.5 wt. %, further optionally about 0.1 wt. % to about 0.3 wt. % and in some embodiments in various embodiments from about 0.01 wt. % to about 1 wt. %, from about 0.05 to about 2%, from about 0.1% to about 2.5%, and from about 0.1 to about 0.5% by total weight of the composition.

In some embodiments, the oral care compositions comprise at least one sweetener, useful for example to enhance taste of the composition. Sweetening agents among those useful herein include dextrose, polydextrose, sucrose, maltose, dextrin, dried invert sugar, mannose, xylose, ribose, fructose, levulose, galactose, corn syrup, partially hydrolyzed starch, hydrogenated starch hydrolysate, ethanol, sorbitol, mannitol, xylitol, maltitol, isomalt, aspartame, neotame, saccharin and salts thereof (e.g. sodium saccharin), sucralose, dipeptide-based intense sweeteners, cyclamates, dihydrochalcones, glycerine, propylene glycol, polyethylene glycols, Poloxomer polymers such as POLOXOMER 407, PLURONIC F108, (both available from BASF Corporation), alkyl polyglycoside (APG), polysorbate, PEG40, castor oil, menthol, and mixtures thereof. One or more sweeteners are optionally present in a total amount depending strongly on the particular sweetener(s) selected, but typically 0.005 wt. % to 5 wt. %, by total weight of the composition, optionally 0.005 wt. % to 0.2 wt. %, further optionally 0.05 wt. % to 0.1 wt. % by total weight of the composition.

In some embodiments, the oral care compositions further comprise an agent that interferes with or prevents bacterial attachment, e.g., ethyl lauroyl arginiate (ELA), solbrol or chitosan, as well as plaque dispersing agents such as enzymes (papain, glucoamylase, etc.).

In some embodiments, the oral care compositions also comprise at least one surfactant. Any orally acceptable surfactant, most of which are anionic, cationic, zwitterionic, nonionic or amphoteric, and mixtures thereof, can be used. Examples of suitable surfactants include water-soluble salts of higher fatty acid monoglyceride monosulfates, such as the sodium salt of monosulfated monoglyceride of hydrogenated coconut oil fatty acids; higher alkyl sulfates such as sodium lauryl sulfate, sodium coconut monoglyceride sulfonate, sodium lauryl sarcosinate, sodium lauryl isoethionate, sodium laureth carboxylate and sodium dodecyl benzenesulfonate; alkyl aryl sulfonates such as sodium dodecyl benzene sulfonate; higher alkyl sulfoacetates, such as sodium lauryl sulfoacetate; higher fatty acid esters of 1,2-dihydroxypropane sulfonate; and the substantially saturated higher aliphatic acyl amides of lower aliphatic amino carboxylic compounds, such as those having 12-16 carbons in the fatty acid, alkyl or acyl radicals; and the like. Examples of amides include N-lauryl sarcosine, and the sodium, potassium and ethanolamine salts of N-lauryl, N-myristoyl, or N-palmitoyl sarcosine. Examples of cationic surfactants include derivatives of aliphatic quaternary ammonium compounds having one long alkyl chain containing 8 to 18 carbon atoms such as lauryl trimethylammonium chloride, cetyl pyridinium chloride, cetyl trimethyl ammonium bromide, di-isobutylphenoxyethyldimethylbenzylammonium chloride, coconut alkyltrimethylammonium nitrite, cetyl pyridinium fluoride, and mixtures thereof. Suitable nonionic surfactants include without limitation, poloxamers, polyoxyethylene sorbitan esters, fatty alcohol ethoxylates, alkylphenol ethoxylates, tertiary amine oxides, tertiary phosphine oxides, di alkyl sulfoxides and the like. Others include, for example, non-anionic polyoxyethylene surfactants, such as Polyoxamer 407, Steareth 30, Polysorbate 20, and castor oil; and amphoteric surfactants such as derivatives of aliphatic secondary and tertiary amines having an anionic group such as carboxylate, sulfate, sulfonate, phosphate or phosphonate such as cocamidopropyl betaine (tegobaine), and cocamidopropyl betaine lauryl glucoside; condensation products of ethylene oxide with various hydrogen containing compounds that are reactive therewith and have long hydrocarbon chains (e.g., aliphatic chains of from 12 to 20 carbon atoms), which condensation products (ethoxamers) contain hydrophilic polyoxyethylene moieties, such as condensation products of poly (ethylene oxide) with fatty acids, fatty, alcohols, fatty amides and other fatty moieties, and with propylene oxide and polypropylene oxides. In some embodiments, the oral composition includes a surfactant system that is sodium laurel sulfate (SLS) and cocamidopropyl betaine. One or more surfactants are optionally present in a total amount of about 0.01 wt. % to about 10 wt. %, for example, from about 0.05 wt. % to about 5 wt. %, or from about 0.1 wt. % to about 2 wt. %, e.g., 1.5% wt. by total weight of the composition. In some embodiments, the oral composition includes an anionic surfactant, e.g., a surfactant selected from sodium lauryl sulfate, sodium ether lauryl sulfate, and mixtures thereof, e.g., in an amount of from about 0.3% to about 4.5% by weight, e.g., 1-2% sodium lauryl sulfate (SLS); and/or a zwitterionic surfactant, for example a betaine surfactant, for example cocamidopropyl betaine, e.g., in an amount of from about 0.1% to about 4.5% by weight, e.g., 0.5-2% cocamidopropylbetaine. Some embodiments comprise a nonionic surfactant in an amount of from 0.5-5%, e.g., 1-2%, selected from poloxamers (e.g., poloxamer 407), polysorbates (e.g., polysorbate 20), polyoxyl hydrogenated castor oil (e.g., polyoxyl 40 hydrogenated castor oil), and mixtures thereof. In some embodiments, the poloxamer nonionic surfactant has a polyoxypropylene molecular mass of from 3000 to 5000 g/mol and a polyoxyethylene content of from 60 to 80 mol %, e.g., the poloxamer nonionic surfactant comprises poloxamer 407. Any of the preceding compositions may further comprise sorbitol, wherein the sorbitol is in a total amount of 10-40% (e.g., about 23%).

In some embodiments, the oral care compositions comprise at least, one foam modulator, useful for example to increase amount, thickness or stability of foam generated by the composition upon agitation. Any orally acceptable foam modulator can be used, including without limitation, polyethylene glycols (PEGs), also known as polyoxyethylenes. High molecular weight PEGS are suitable, including those having an average molecular weight of 200,000 to 7,000,000, for example 500,000 to 5,000,000, or 1,000,000 to 2,500,000, One or more PEGS are optionally present in a total amount of about 0.1 wt. % to about 10 wt. %, for example from about 0.2 wt. % to about 5 wt. %, or from about 0.25 wt. % to about 2 wt. %, by total weight of the composition

In some embodiments, the oral care compositions comprise at least one pH modifying agent. Such agents include acidifying agents to lower pH, basifying agents to raise pH, and buffering agents to control pH within a desired range. For example, one or more compounds selected from acidifying, basifying and buffering agents can be included to provide a pH of 2 to 10, or in various illustrative embodiments, 2 to 8, 3 to 9, 4 to 8, 5 to 7, 6 to 10, 7 to 9, etc. Any orally acceptable pH modifying agent can be used, including without limitation, carboxylic, phosphoric and sulfonic acids, acid salts (e.g., monosodium citrate, disodium citrate, monosodium malate, etc.), alkali metal hydroxides such as sodium hydroxide, carbonates such as sodium carbonate, bicarbonates such as sodium bicarbonate, sesquicarbonates, borates, silicates, bisulfates, phosphates (e.g., monosodium phosphate, trisodium phosphate, monopotassium phosphate, dipotassium phosphate, tribasic sodium phosphate, sodium tripolyphosphate, phosphoric acid), imidazole, sodium phosphate buffer (e.g., sodium phosphate monobasic and disodium phosphate) citrates (e.g. citric acid, trisodium citrate dehydrate), pyrophosphates (sodium and potassium salts) and the like and combinations thereof. One or more pH modifying agents are optionally present in a total amount effective to maintain the composition in an orally acceptable pH range. Compositions may have a pH that is either acidic or basic, e.g., from pH 4 to pH 5.5 or from pH 8 to pH 10. In some embodiments, the amount of buffering agent is sufficient to provide a pH of about 5 to about 9, preferable about 6 to about 8, and more preferable about 7, when the composition is dissolved in water, a mouth rinse base, or a toothpaste base. Typical amounts of buffering agent are about 5% to about 35%, in one embodiment about 10% to about 30%), in another embodiment about 15% to about 25%, by weight of the total composition.

In some embodiments, the oral care compositions also comprise at least one humectant. Any orally acceptable humectant can be used, including without limitation, polyhydric alcohols such as glycerin, sorbitol (optionally as a 70 wt. % solution in water), propylene glycol, xylitol or low molecular weight polyethylene glycols (PEGS) and mixtures thereof. Most humectants also function as sweeteners. In some embodiments, compositions comprise 15% to 70% or 30% to 65% by weight humectant. Suitable humectants include edible polyhydric alcohols such as glycerine, sorbitol, xylitol, propylene glycol as well as other polyols and mixtures of these humectants. Mixtures of glycerine and sorbitol may be used in certain embodiments as the humectant component of the compositions herein. One or more humectants are optionally present in a total amount of from about 1 wt. % to about 70 wt. %, for example, from about 1 wt. % to about 50 wt. %, from about 2 wt. % to about 25 wt. %, or from about 5 wt. % to about 15 wt. %, by total weight of the composition. In some embodiments, humectants, such as glycerin are present in an amount that is at least 20%>, e.g., 20-40%, e.g., 25-35%.

Mouth-feel agents include materials imparting a desirable texture or other feeling during use of the composition. In some embodiments, the oral care compositions comprise at least one thickening agent, useful for example to impart a desired consistency and/or mouth feel to the composition. Any orally acceptable thickening agent can be used, including without limitation, carbomers, also known as carboxyvinyl polymers, carrageenans, also known as Irish moss and more particularly i-carrageenan (iota-carrageenan), cellulosic polymers such as hydroxyethyl cellulose, and water-soluble salts of cellulose ethers (e.g., sodium carboxymethyl cellulose and sodium carboxymethyl hydroxyethyl cellulose), carboxymethylcellulose (CMC) and salts thereof, e.g., CMC sodium, natural gums such as karaya, xanthan, gum arabic and tragacanthin, colloidal magnesium aluminum silicate, colloidal silica, starch, polyvinyl pyrrolidone, hydroxyethyl propyl cellulose, hydroxybutyl methyl cellulose, hydroxypropyl methyl cellulose, and hydroxyethyl cellulose and amorphous silicas, and the like. A preferred class of thickening or gelling agents includes a class of homopolymers of acrylic acid crosslinked with an alkyl ether of pentaerythritol or an alkyl ether of sucrose, or carbomers. Carbomers are commercially available from B. F. Goodrich as the Carbopol© series. Particularly preferred Carbopols include Carbopol 934, 940, 941, 956, 974P, and mixtures thereof. Silica thickeners such as DT 267 (from PPG Industries) may also be used. One or more thickening agents are optionally present in a total amount of from about 0.01 wt. % to 15 wt. %, for example from about 0.1 wt. % to about 10 wt. %, or from about 0.2 wt. % to about 5 wt. %, by total weight of the composition. Some embodiments comprise sodium carboxymethyl cellulose (e.g., from 0.5 wt. %-1.5 wt. %). In certain embodiments, thickening agents in an amount of about 0.5% to about 5.0% by weight of the total composition are used. Thickeners may be present in an amount of from 1 wt % to 15 wt %, from 3 wt % to 10 wt %, 4 wt % to 9 wt %, from 5 wt % to 8 wt %, for example 5 wt %, 6 wt %, 7 wt %, or 8 wt %.

In some embodiments, the oral care compositions comprise at least one colorant. Colorants herein include pigments, dyes, lakes and agents imparting a particular luster or reflectivity such as pearling agents. In various embodiments, colorants are operable to provide a white or light-colored coating on a dental surface, to act as an indicator of locations on a dental surface that have been effectively contacted by the composition, and/or to modify appearance, in particular color and/or opacity, of the composition to enhance attractiveness to the consumer. Any orally acceptable colorant can be used, including FD&C dyes and pigments, talc, mica, magnesium carbonate, calcium carbonate, magnesium silicate, magnesium aluminum silicate, silica, titanium dioxide, zinc oxide, red, yellow, brown and black iron oxides, ferric ammonium ferrocyanide, manganese violet, ultramarine, titaniated mica, bismuth oxychloride, and mixtures thereof. One or more colorants are optionally present in a total amount of about 0.001% to about 20%, for example about 0.01% to about 10% or about 0.1% to about 5% by total weight of the composition.

In some embodiments, the oral care composition further comprises an anti-calculus (tartar control) agent. Suitable anti-calculus agents include, but are not limited to: phosphates and polyphosphates, polyaminopropane sulfonic acid (AM PS), polyolefin sulfonates, polyolefin phosphates, diphosphonates such as azacycloalkane-2,2-diphosphonates (e.g., azacycloheptane-2,2-diphosphonic acid), N-methyl azacyclopentane-2,3-diphosphonic acid, ethane-1-hydroxy-1,1-diphosphonic acid (EHDP) and ethane-1-amino-1,1-diphosphonate, and phosphonoalkane carboxylic acids. Useful inorganic phosphate and polyphosphate salts include monobasic, dibasic and tribasic sodium phosphates. Soluble pyrophosphates are useful anticalculus agents. The pyrophosphate salts can be any of the alkali metal pyrophosphate salts. In certain embodiments, salts include tetra alkali metal pyrophosphate, dialkali metal diacid pyrophosphate, trialkali metal monoacid pyrophosphate and mixtures thereof, wherein the alkali metals are sodium or potassium. The pyrophosphates also contribute to preservation of the compositions by lowering water activity, tetrasodium pyrophosphate (TSPP), tetrapotassium pyrophosphate, sodium tripolyphosphate, tetrapolyphosphate, sodium trimetaphosphate, sodium hexametaphosphate and mixtures thereof. The salts are useful in both their hydrated and unhydrated forms. An effective amount of pyrophosphate salt useful in the present composition is generally enough to provide least 0.1 wt. % pyrophosphate ions, e.g., 0.1 to 3 wt. %, e.g., 0.1 to 2 wt. %, e.g., 0.1 to 1 wt. %, e.g., 0.2 to 0.5 wt. %.

Other useful tartar control agents include polymers and co-polymers. In some embodiments, the oral care compositions include one or more polymers, such as polyethylene glycols, polyvinyl methyl ether maleic acid copolymers, polysaccharides (e.g., cellulose derivatives, for example carboxymethyl cellulose, or polysaccharide gums, for example xanthan gum or carrageenan gum). Acidic polymers, for example polyacrylate gels, may be provided in the form of their free acids or partially or fully neutralized water-soluble alkali metal (e.g., potassium and sodium) or ammonium salts. Certain embodiments include 1:4 to 4:1 copolymers of maleic anhydride or acid with another polymerizable ethylenically unsaturated monomer, for example, methyl vinyl ether (methoxyethylene), having a molecular weight (M.W.) of about 30,000 to about 1,000,000, polyvinyl methyl ether/maleic anhydride (PVM/MA) copolymers such as GANTREZ® (e.g., GANTREZ® S-97 polymer). In some embodiments, the PVM/MA copolymer comprises a copolymer of methyl vinyl ether/maleic anhydride, wherein the anhydride is hydrolyzed following copolymerization to provide the corresponding acid. In some embodiments, PVM/MA copolymer has an average molecular weight (M.W.) of about 30,000 to about 1,000,000, e.g., about 300,000 to about 800,000, e.g., wherein the anionic polymer is about 1-5%, e.g., about 2%, of the weight of the composition. In some embodiments, the anti-calculus agent is present in the composition in an amount of from 0.2 weight % to 0.8 weight %; 0.3 weight % to 0.7 weight %; 0.4 weight % to 0.6 weight %; or about 0.5 weight %, based on the total weight of the composition. Copolymers are available for example as Gantrez AN 139 (M.W. 500,000), AN 1 19 (M.W. 250,000) and S-97 Pharmaceutical Grade (M.W. 70,000), of GAF Chemicals Corporation. Other operative polymers include those such as the 1:1 copolymers of maleic anhydride with ethyl acrylate, hydroxyethyl methacrylate, N-vinyl-2-pyrollidone, or ethylene, the latter being available for example as Monsanto EMA No. 1 103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone. Suitable generally, are polymerized olefinically or ethyl enically unsaturated carboxylic acids containing an activated carbon-to-carbon olefinic double bond and at least one carboxyl group, that is, an acid containing an olefinic double bond which readily functions in polymerization because of its presence in the monomer molecule either in the alpha-beta position with respect to a carboxyl group or as part of a terminal methylene grouping. Illustrative of such acids are acrylic, methacrylic, ethacrylic, alpha-chloroacrylic, crotonic, beta-acryloxy propionic, sorbic, alpha-chlorsorbic, cinnamic, beta-styrylacrylic, muconic, itaconic, citraconic, mesaconic, glutaconic, aconitic, alpha-phenylacrylic, 2-benzyl acrylic, 2-cyclohexylacrylic, angelic, umbellic, fumaric, maleic acids and anhydrides. Other different olefinic monomers copolymerizable with such carboxylic monomers include vinylacetate, vinyl chloride, dimethyl maleate and the like. Copolymers contain sufficient carboxylic salt groups for water-solubility. A further class of polymeric agents includes a composition containing homopolymers of substituted acrylamides and/or homopolymers of unsaturated sulfonic acids and salts thereof, in particular where polymers are based on unsaturated sulfonic acids selected from acrylamidoalykane sulfonic acids such as 2-acrylamide 2 methylpropane sulfonic acid having a molecular weight of about 1,000 to about 2,000,000. Another useful class of polymeric agents includes polyamino acids, particularly those containing proportions of anionic surface-active amino acids such as aspartic acid, glutamic acid and phosphoserine.

In some embodiments, the oral care compositions comprise a saliva stimulating agent useful, for example, in amelioration of dry mouth. Any orally acceptable saliva stimulating agent can be used, including without limitation food acids such as citric, lactic, malic, succinic, ascorbic, adipic, fumaric and tartaric acids, and mixtures thereof. One or more saliva stimulating agents are optionally present in saliva stimulating effective total amount.

In some embodiments, the oral care compositions comprise a nutrient. Suitable nutrients include vitamins, minerals, amino acids, and mixtures thereof. Vitamins include Vitamins C and D, miamine, riboflavin, calcium pantothenate, niacin, folic acid, nicotinamide, pyridoxine, cyanocobalamin, para-aminobenzoic acid, bioflavonoids, and mixtures thereof. Nutritional supplements include amino acids (such as L-tryptophane, L-lysine, methionine, threonine, levocarnitine and L-carnitine), lipotropics (such as choline, inositol, betaine, and linoleic acid), and mixtures thereof.

In some embodiments, the oral care compositions comprise at least one viscosity modifier, useful for example to help inhibit settling or separation of ingredients or to promote re-dispersibility upon agitation of a liquid composition. Any orally acceptable viscosity modifier can be used, including without limitation, mineral oil, petrolatum, clays and organo-modified clays, silicas and the like. One or more viscosity modifiers are optionally present in a total amount of from about 0.01 wt. % to about 10 wt. %, for example, from about 0.1 wt. % to about 5 wt. %, by total weight of the composition.

In some embodiments, the oral care compositions comprise antisensitivity agents, e.g., potassium salts such as potassium nitrate, potassium bicarbonate, potassium chloride, potassium citrate, and potassium oxalate; capsaicin; eugenol; strontium salts; chloride salts and combinations thereof. Such agents may be added in effective amounts, e.g., from about 1 wt. % to about 20 wt. % by weight based on the total weight of the composition, depending on the agent chosen.

In some embodiments, the oral care compositions comprise an antioxidant. Any orally acceptable antioxidant can be used, including butylated hydroxy anisole (BHA), butylated hydroxytoluene (BHT), vitamin A, carotenoids, co-enzyme Q10, PQQ, Vitamin A, Vitamin C, vitamin E, anethole-dithiothione, flavonoids, polyphenols, ascorbic acid, herbal antioxidants, chlorophyll, melatonin, and mixtures thereof.

In some embodiments, the oral care compositions comprise of one or more alkali phosphate salts, e.g., sodium, potassium or calcium salts, e.g., selected from alkali dibasic phosphate and alkali pyrophosphate salts, e.g., alkali phosphate salts selected from sodium phosphate dibasic, potassium phosphate dibasic, dicalcium phosphate dihydrate, calcium pyrophosphate, tetrasodium pyrophosphate, tetrapotassium pyrophosphate, sodium tripolyphosphate, disodium hydrogenorthophoshpate, monosodium phosphate, pentapotassium triphosphate and mixtures of any of two or more of these, e.g., in an amount of 0.01-20%, e.g., 0.1-8%, e.g., e.g., 0.1 to 5%, e.g., 0.3 to 2%, e.g., 0.3 to 1%, e.g about 0.01%, about 0.1%, about 0.5%, about 1%, about 2%, about 5%, about 6%, by weight of the composition. In some embodiments, compositions comprise tetrapotassium pyrophosphate, disodium hydrogenorthophoshpate, monosodium phosphate, and pentapotassium triphosphate. In some embodiments, compositions comprise tetrasodium pyrophosphate from 0.1-1.0 wt % (e.g., about 0.5 wt %).

In some embodiments, the oral care compositions comprise a source of calcium and phosphate selected from (i) calcium-glass complexes, e.g., calcium sodium phosphosilicates, and (ii) calcium-protein complexes, e.g., casein phosphopeptide-amorphous calcium phosphate. Any of the preceding compositions further comprising a soluble calcium salt, e.g., selected from calcium sulfate, calcium chloride, calcium nitrate, calcium acetate, calcium lactate, and combinations thereof.

In some embodiments, the oral care compositions comprise an additional ingredient selected from: benzyl alcohol, Methylisothizolinone (“MIT”), Sodium bicarbonate, sodium methyl cocoyl taurate (tauranol), lauryl alcohol, and polyphosphate. Some embodiments comprise benzyl alcohol that is present from 0.1-0.8 wt %., or 0.2 to 0.7 wt %, or from 0.3 to 0.6 wt %, or from 0.4 to 0.5 wt %, e.g., about 0.1 wt. %, about 0.2 wt. %, about 0.3 wt %, about 0.4 wt %, about 0.5 wt %, about 0.6 wt %, about 0.7 wt % or about 0.8 wt %.

In some embodiments, the oral care compositions comprise from 5%-40%, e.g., 10%-35%, e.g., about 15%, 25%, 30%, and 35% or more of water.

### EXAMPLES

### Example 1

A study was conducted to assess the potential oral health benefits of a toothpaste that comprises Zinc Citrate, Zinc Oxide and Arginine. The effect on the level of sIgA in a sample from buccal surface by the use of a Testing toothpaste (about 1.0 wt %. zinc oxide, about 0.5 wt %. zinc citrate trihydrate, about 1.5 wt % L-arginine) twice daily for seven consecutive days was compared to the effect on sIgA levels by the use of a Control toothpaste (containing no Zinc Citrate, Zinc Oxide and Arginine) following same application regimen. SIga levels were measured using an ELISA assay on Days 0, 3 and 7.

Details of Procedure:

1. Study Subjects: Using Inclusion/Exclusion Criteria set forth below, 72 subjects were recruited for the study. Subjects provided informed consent.

2. Baseline buccal cell collection: Subjects were asked to refrain from all oral hygiene procedures, eating, drinking, or smoking for one hour prior to the visit. Cheek buccal cells will be collected from the mouth by using a cotton swab. Cells were re-suspended in 2 tubes containing 1 ml of PBS each for sIgA analysis.

3. Toothpaste usage: Randomly grouped subjects were assigned one dentifrice and a soft-bristled toothbrush to use at home. Testing Toothpaste included ZnO, ZnCl₂, arginine, sodium fluoride, abrasives, flavorants, sweeteners, surfactants, pH modifying agents, humectants, thickening agents, colorant, benzyl alcohol and water. Control Toothpaste did not contain ZnO, ZnCl₂ and Arg. Subjects were instructed to brush their teeth twice daily (in the morning and evening) for two minutes. They were instructed to cover the entire length (head) of the toothbrush bristles with a ribbon of their assigned dentifrice.

4. Instructions to Subjects: Subjects were instructed to use only the study products during the seven-day usage period. Subjects were instructed to refrain from using any other oral hygiene products such as other dentifrices or toothbrushes, mouth rinses, dental flosses, and interdental stimulators as well as any anti-inflammation drugs while they were using one of the study products. Subjects were also be instructed to refrain from smoking during the study. Subject were instructed to come back to collect buccal cells after 3 days, 7 days and 14 days usage of toothpaste.

5. Buccal cells collection at 3 days, 7 days and 14 days: After 3 days, 7 days and 14 days usage of the toothpaste, buccal cells were collected. Subjects were asked to refrain from all oral hygiene procedures, eating, drinking, or smoking for one hour prior to the visit. Cheek buccal cells were collected from the mouth by using a cotton swab. Cells were re-suspended in 2 tubes containing 1 ml of PBS each for sIgA analysis.

6. SIgA Analysis:
1) Qualification study of sIgA by fluorescence microscopy
   a) One tube of buccal cells sample from each time point were taken and spun down at 800 g for 5 mins.
   b) Cells in pellet were re-suspended in 1 ml PBS and spun down at 800 g for 5 mins
   c) Cells were re-suspended in 96 μl of PBS containing 3 μl of Anti-Human IgA (a-chain specific)-FITC (Sigma cat #F5259) (32:1 dilution) and maintained at room temperature for one hour
   d) Cells were washed with 3 times using 1 ml of PBS and cells were re-suspend in 50 μl of PBS. Cell samples were transferred on a slide and a photographic image was made under fluorescence microscopy (excitation: 495 nm and emission: 519 nm)
2) Quantification study of SIgA by Enzyme Linked Immunosorbent Assay (ELISA): sIgA was quantitatively measured by ELISA Kit (ab137980-IgA Human ELISA Kit) from Abcam

Statistical Analysis

The comparisons on SIgA in two groups was performed using Minitab 16 statistical software. Data was analyzed by Tukey Method and 95.0% Confidence level.

Inclusion/Exclusion Criteria

All subjects were enrolled into the study based on following criteria
Subjects have to be at least 18 years of age, in general good health, and possess at least 20 natural uncrowned teeth.
Subjects have to be available for the duration of the seven-day study and sign an Informed Consent Form.
Subjects will be excluded from the study if they have participated in any other dental study within three months prior to the start of the clinical study.
Subjects will be excluded from the study if they have systemic conditions (AIDS, renal, hepatic or heart disease) or take any prescription medications.
Subjects will be excluded from the study if they have a history of allergies to oral care products, personal care consumer products or their ingredients.
Subjects will be excluded from the study if they are pregnant, expecting to be pregnant or lactating.

Results

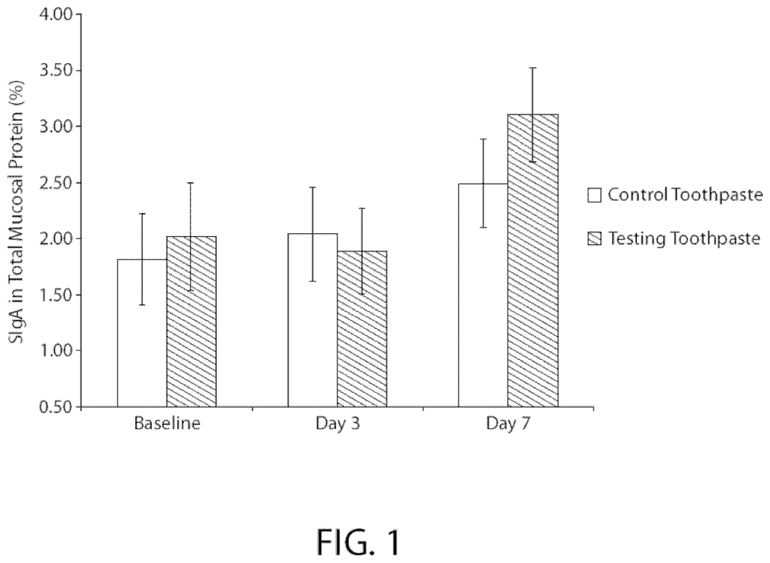

Data is shown in FIG. 1. Results of the study showed that when applied twice daily for seven consecutive day, a significantly higher increase of sIgA level was observed compared to the level observed using a control toothpaste. The significantly higher increase of sIgA produced by the use of the oral care composition comprising Zinc Oxide, Zinc Citrate and Arginine twice daily for seven consecutive days demonstrates that when used twice daily for seven consecutive days, the oral care composition boosted natural defenses in the oral cavity significantly. Day 3 results indicate that sIgA levels were substantially unchanged from baseline.

### Example 2

An investigation was undertaken assess the potential oral health benefits of a toothpaste that comprises Zinc Citrate, Zinc Oxide and Arginine. The effect on the level of sIgA on cells in a sample from buccal/gum surface which contains oral epithelial cells surface including the mucosa pellicle following use of a Test toothpaste (about 1.0 wt %. zinc oxide, about 0.5 wt %. zinc citrate, about 5.0 wt % arginine) compared to the effect on sIgA levels on sample cells following use of a Control toothpaste (commercially available fluoride toothpaste containing no Zinc Citrate, Zinc Oxide and Arginine). Cells from buccal/gum sample were stained for Zinc and for IgA following contact with either the Test toothpaste or Control toothpaste.

Procedure:

Treatment and Sample Collection

Before brushing, buccal/gum surfaces were swabbed to collect cells from right side of the mouth. Cells from this sample were isolated and stained for either zinc of IgA. The staining results for this sample provide a baseline for comparison with cells that obtained after brushing with Test toothpaste and stained and with cells that obtained after brushing with control toothpaste and stained.

Subjects then brushed teeth with either the Test toothpaste (which contains zinc oxide, zinc citrate, arginine, sodium fluoride, abrasives, flavorants, sweeteners, surfactants, pH modifying agents, humectants, thickening agents, colorant, anti-calculus agent and water) or a commercially available fluoride toothpaste that does not contain zinc or arginine. Brushing was done for 1 min followed by swishing slurry around in mouth for 30 s and rinsing.

Immediately after brushing, buccal/gum surfaces were swabbed to collect cells. Cells from the sample taken from subjects who brushed with the Test toothpaste were isolated and stained for either zinc of IgA. The staining results for this sample were compared with cells that were stained before brushing and cells stained one hour after brushing with for comparison with cells that obtained after brushing and stained.

One hour after brushing, buccal/gum surfaces were swabbed to collect cells from right side of the mouth. Cells from the sample taken from subjects who brushed with the Test toothpaste were isolated and stained for either zinc of IgA. Likewise, cells from the sample taken from subjects who brushed with the control toothpaste were isolated and stained for either zinc of IgA. The staining results for this sample were compared with cells that were stained before brushing and cells taken immediately after brushing with test toothpaste stained.

Stain Cells for Zinc

Cells from the sample taken before brushing, cells from the sample taken immediately after brushing with Test toothpaste, cells from the sample taken one hour after brushing with Test toothpaste and cells from the sample taken one hour after brushing with control toothpaste were each isolated and stained for Zinc. Cells in each sample were washed with buffer briefly. Cells were collected as a pellet and resuspend in 1000 of buffer containing 2.4 μM of Zinquin. Cells were maintained in buffer-Zinquin mixture for 30 minutes at 37° C. After 30 minutes elapsed, cells were washed three times. In each wash, 1 ml of buffer was added to cells, which were pelleted and resuspended in 500 of buffer. Following final wash, cells were observed under fluorescence microscopy at excitation: 368 nm and emission: 490 nm and photographs were made.

Stain Cells for IgA

Cells from the sample taken before brushing, cells from the sample taken immediately after brushing with Test toothpaste, cells from the sample taken one hour after brushing with Test toothpaste and cells from the sample taken one hour after brushing with control toothpaste were each isolated and stained for IgA. Cells in each sample were washed with buffer briefly. Cells were collected as a pellet and resuspend in 96 μl of buffer containing 3 μl of anti-human IgA (a-chain specific)-FITC (Sigma cat #F5259) (32:1 dilution). Cells were maintained in buffer-anti-human IgA (a-chain specific)-FITC mixture for one hour minutes at 37° C. After one hour elapsed, cells were washed three times. In each wash, 1 ml of buffer was added to cells, which were pelleted and resuspended in 500 of buffer. Following final wash, cells were observed under fluorescence microscopy at excitation: 495 nm and emission: 519 nm and photographs were made.

Results:

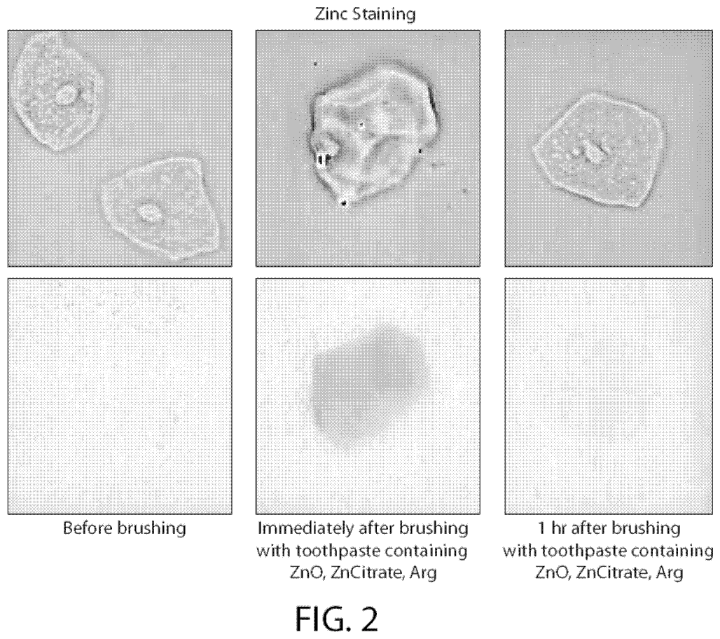

FIG. 2 shows results of zinc staining of oral epithelial cells from samples taken before brushing, immediately after brushing, and one hour after brushing with Test toothpaste. Zinc signal appears blue on cells stained with Zinquin appear under fluorescence microscopy at excitation: 368 nm and emission: 490 nm. As shown in FIG. 2, zinc stained strongly on oral epithelial cells from samples taken immediately after brushing with Test toothpaste is very strong on cells. The blue stain on these cells is very visible. One hour after brushing, blue can faintly be detected compared to baseline.

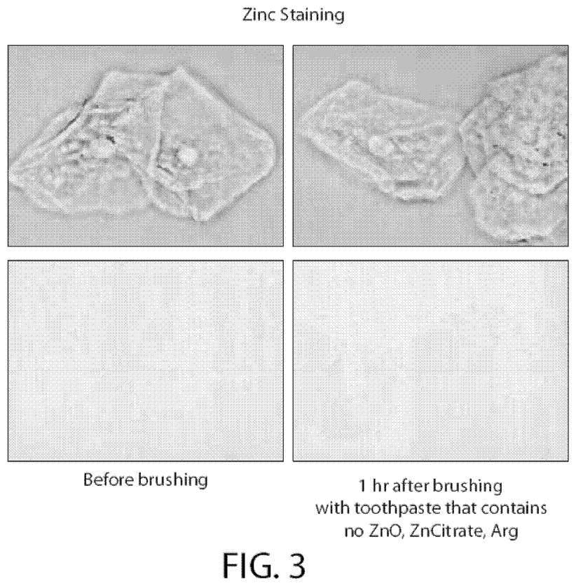

FIG. 3 shows results of zinc staining of oral epithelial cells from samples taken before brushing and one hour after brushing. Zinc signal appears blue on cells stained with Zinquin appear under fluorescence microscopy at 368 nm and emission: 490 nm. As shown in FIG. 3, zinc stained is not detected on oral epithelial cells from samples taken one hour after brushing with control toothpaste.

Zinc staining results demonstrate that cells from subjects who brushed with Test toothpaste showed evidence of zinc on their cells, even on cells one hour after brushing. As expected, cells from subjects who brushed with control toothpaste, which does not contain zinc, showed no evidence of zinc on their cells

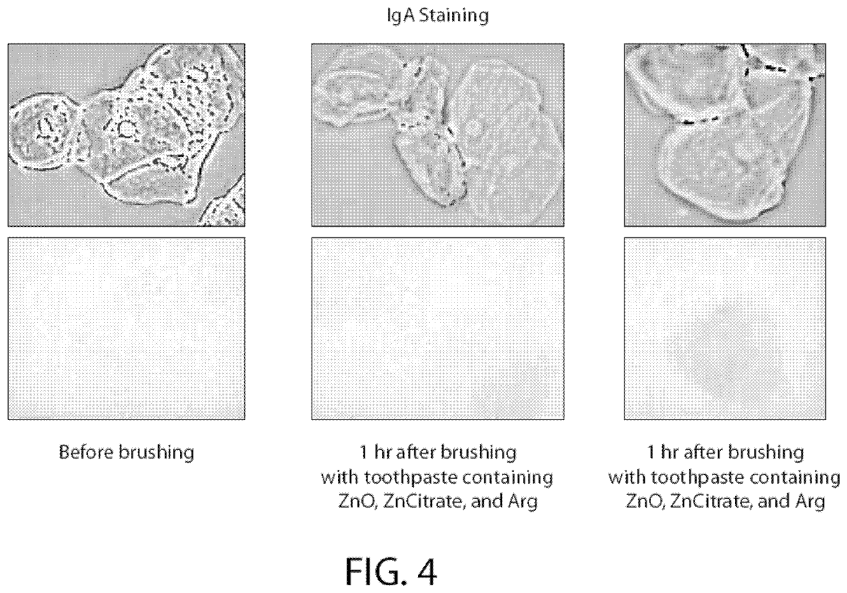

FIG. 4 shows results of IgA staining of oral epithelial cells from samples taken before brushing (left panel), and one hour after brushing with Test toothpaste (center panel and right panel). IgA signal appears green on cells stained with anti-human IgA (a-chain specific)-FITC appear under fluorescence microscopy at excitation: 495 nm and emission: 519 nm. As shown in FIG. 4, IgA is detected in samples taken one hour after brushing with Test toothpaste. The green stain in the right panel is very visible on the cells from the one hour post-brushing while baseline shows no staining. The green fluorescence in the central panel, which is also the 1 hr post brushing, is not strong as the right one. When observed with a microscope, IgA signal is visible.

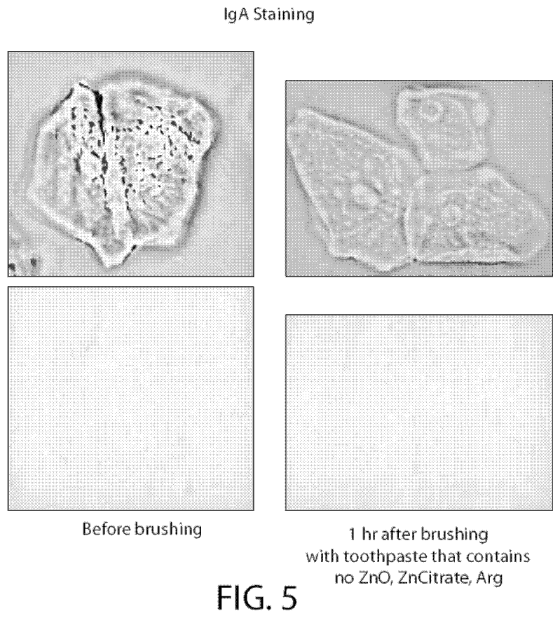

FIG. 5 shows results of IgA staining of oral epithelial cells from samples taken before brushing and one hour after brushing with control toothpaste. IgA signal appears green on cells stained with anti-human IgA (a-chain specific)-FITC appear under fluorescence microscopy at excitation: 495 nm and emission: 519 nm. As shown in FIG. 5, IgA is not detected on oral epithelial cells from samples taken one hour after brushing with control toothpaste.

Conclusion

Zinc was delivered to the oral surface after brushing with Test toothpaste, which contains zinc oxide, zinc citrate and arginine. Brushing with the Test toothpaste, promoted the IgA concentrated on the oral surface. Commercially available fluoride toothpaste does not deliver zinc to the oral surface and there is no evidence that IgA levels on the oral surface change after using control toothpaste.

The increase concentration of IgA on the oral epithelial cells surface that occurs following the use of the oral care composition that comprises zinc oxide, zinc citrate and arginine strengthens the natural defense components in the mucosa pellicle.

### Example 3

Phagocytosis is a very important physiological process which is characterized by the ingestion of foreign particles and killing of microorganisms by phagocytic leukocytes (monocytes, macrophages, granulocytes). The phagocytic leukocytes are part of a first line of natural defense against potential pathogens. Phagocytotic active of phagocytes can be measured using a phagocytosis assay. The phagocytosis assay uses fluorescently-opsonized latex beads which are contacted with phagocytic cells. After maintaining the beads in contact with the cell, fluorescent reading of cells is used to assess the degree of phagocytosis. Thus, the degree of phagocytosis from cells treated with different compounds can be compared quantifiably. The more fluorescence the more active of phagocytosis.

The effect of zinc on two different phagocytotic immune cells lines were investigated. Phagocytotic immune cells were contacted with fluorescently-opsonized latex beads in the presence of zinc citrate, zinc oxide or a combination of zinc oxide in combination with zinc citrate to assess the effects of these ingredients on the phagocytotic activity of phagocytotic immune cells.

EDTA chelates Zinc. Phagocytotic immune cells were contacted with fluorescently-opsonized latex beads in the presence of ZnCl₂ and EDTA to determine the effect addition of zinc-chelating EDTA changes the effects of zinc on the phagocytotic activity of phagocytotic immune cells.

Methods

Preparation of Cells

96-well plates were seeded with either phagocytic immune cells THP1 (cat #TIB202, ATCC) and U937 (cat #CRL-1593.2, ATCC) at 5×10⁴ cells/well in 100 μl of RPMI-1640 medium containing 10 ng/ml phorbol myristic acid (PMA, Sigma) for 48 hr in a CO₂ incubator at 37° C.

Phagocytosis Assay

Medium was changed with 100 μl/ml of RPMI-1640 (Item #500290, Cayman). U937 cells were treated with ZnCl₂ or ZnCl₂ and EDTA. THP1 cells were treated with ZnO or ZnO plus EDTA. 10 μl of the Latex Beads-Rabbit IgG-FITC solution were added to each well. Plates were mixed gently and cells were incubated in a CO₂ incubator at 37° C. for 24-48 hrs.

After incubation, plates were centrifuged for 5 minutes at 400×g at room temperature. Supernatant was carefully aspirated. 50 μl of Trypan blue solution was added to each well and plates were incubated for 1-2 mins at room temperature. Plates were centrifuged for 5 minutes at 400×g at room temperature. Supernatant was carefully aspirated. Fluorescent intensity was read using in plate reader at excitation 485/emission 535 nm.

Results:

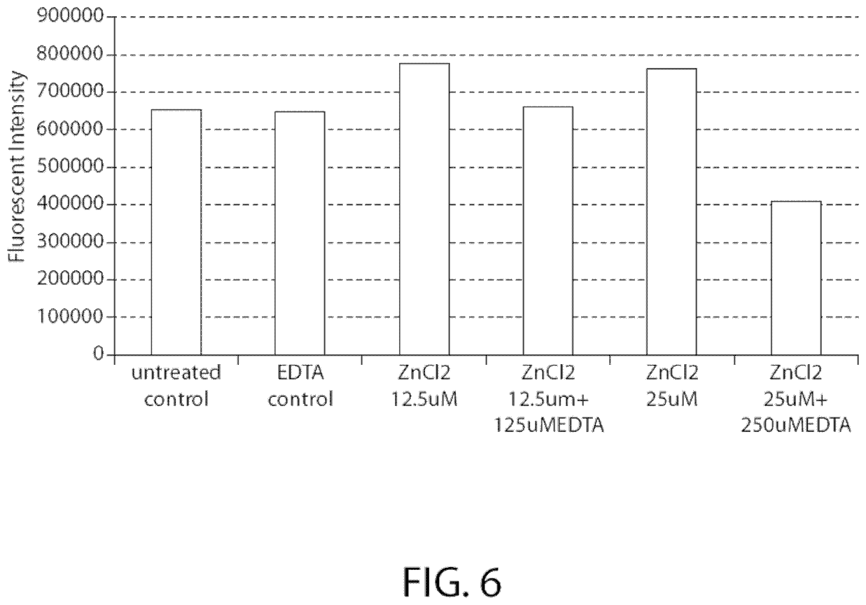

Data from phagocytosis assay using U937 cells and ZnCl2 and ZnCl2 plus EDTA is shown in FIG. 6. No difference in phagocytosis activity was observed between untreated control and EDTA control. ZnCl2 was tested at 12.5 μM and 25 μM. Cells treated with ZnCl₂ at both amounts have more phagocytosis activity than untreated control cells. Addition of EDTA to chelate Zn resulted in a decrease in phagocytosis activity.

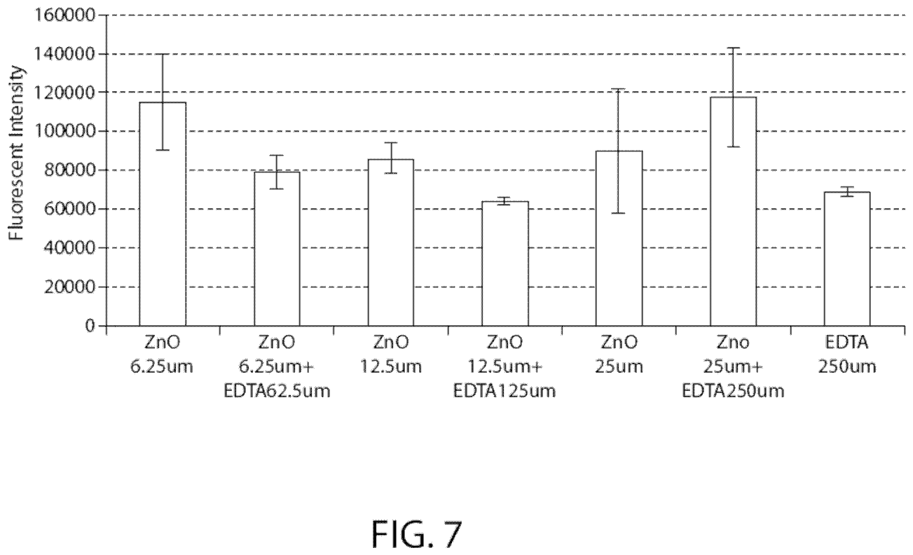

Data from phagocytosis assay using THP1 cells and ZnO and ZnO plus EDTA is shown in FIG. 7. FIG. 7 shows the phagocytosis activity level of EDTA control, which as shown in FIG. 6 is the same level as untreated control. ZnO was tested at 6.25 μM 12.5 μM and 25 μM. Cells treated with ZnO at each amount have more phagocytosis activity than EDTA control cells; ZnO at 6.25 μM showed the greatest increase. Addition of EDTA to chelate Zn resulted in a decrease in phagocytosis activity except in the case of ZnO was tested at 25 μM. ZnO increases phagocytosis activity.

Conclusion: Zinc enhances human natural defense by promoting phagocytosis activity by phagocytotic immune cells.

### Example 4

β-Defensins are cationic peptides with broad-spectrum antimicrobial activity that are produced by epithelia at mucosal surfaces and play an important in the innate defenses against oral microorganisms. The effect of zinc on production β-Defensin levels was investigated using oral epithelial tissue and toothpaste containing zinc oxide.

Procedure:

MatTek EpiOral-FT (ORL-300, MatTek) oral epithelial tissue was used for this test.

100 μl of 1:2 (1 part toothpaste: 2 parts PBS) slurry of the toothpaste was added to the apical surface of the tissue. Two types of toothpastes were used, one that contained ZnO and one that did not contain zinc. After exposure for 2 minutes, tissue samples were washed with PBS 3 times. 6 hrs after the first application of toothpaste/PBS slurry, the treatment was repeated using 100 μl of 1:2 slurry. Tissue samples were rinsed with PBS and incubated for 24 hr, 48 hr and 72 hr. Supernatant was collected at 24 hr, 48 hr and 72 hr for β-defensin 1 measurement.

Results:

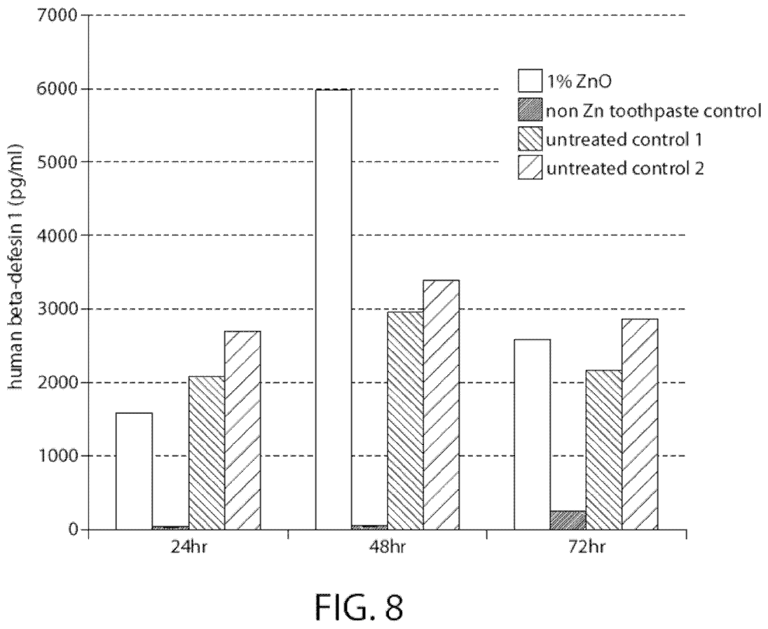

Data is shown in FIG. 8. At 48 hrs after treatment, tissues treated with ZnO containing toothpaste produced significantly more β-defensin 1 than control.

Conclusion:

Zinc enhances human natural defense by promoting oral epithelial tissue secretion of the antimicrobial peptide β-defensin 1.

### Example 5

Oral compositions that comprise arginine are disclosed in WO 2015/094849, which corresponds to US 2016/0338921, which are both incorporated herein by reference. In some embodiments the oral care composition comprises: arginine, in free or salt form; and zinc oxide and zinc citrate. In some embodiments, the arginine is present in an amount of 0.5 weight % to 3 weight %, such as 1 weight % to 2.85 weight %, such as 1.17 weight % to 2.25 weight %, such as 1.4 weight % to 1.6 weight %, such as about 1.5 weight %, based on the total weight of the composition. In some embodiments set out above, the total concentration of zinc salts in the composition is 0.2 weight % to 5 weight %, based on the total weight of the composition. In some embodiments set out above, the molar ratio of arginine to total zinc salts is 0.05:1 to 10:1. In some embodiments set out above, the composition comprises zinc oxide in an amount of 0.5 weight % to 1.5 weight %, such as 1 weight %, and zinc citrate in an amount of 0.25 weight % to 0.75 weight %, such as 0.5 weight %, based on the total weight of the composition. In some embodiments set out above, the weight ratio of zinc oxide to zinc citrate is 1.5:1 to 4.5:1, optionally 1.5:1 to 4:1, 1.7:1 to 2.3:1, 1.9:1 to 2.1:1, or about 2:1.

### Example 6

Oral compositions that comprise arginine are disclosed in WO 2017/003844, which corresponds to US 2018/0021234, which are both incorporated herein by reference. In some embodiments, the oral care composition comprises: arginine, zinc oxide and zinc citrate and a fluoride source. In some embodiments, the arginine has the L-configuration. In some embodiments, the arginine is present in an amount corresponding to 0.1% to 15%, or 0.1% to 8%, or about 5.0 wt. %, or about 8.0 wt. %, or about 1.5 wt. %, based on the total weight of the composition, the weight of the arginine acid being calculated as free form. In some embodiments, the arginine is in free form or partially or wholly salt form. In some embodiments set out above, the ratio of the amount of zinc oxide (by wt %) to zinc citrate (by wt %) is 2:1, 2.5:1, 3:1, 3.5:1 or 4:1, wherein the ratio is by wt. of the overall composition. In some embodiments, the zinc citrate is in an amount of from 0.25 to 1.0 wt % and zinc oxide may be present in an amount of from 0.75 to 1.25 wt % or the zinc citrate is in an amount of about 0.5 wt % and zinc oxide is present in an amount of about 1.0%, based on the total weight of the composition. In some embodiments set out above, the fluoride source is sodium fluoride or sodium monofluorophosphate. In some such embodiments, the sodium fluoride or sodium monofluorophosphate is from 0.1 wt. %-2 wt. % based on the total weight of the composition. In some embodiments, the sodium fluoride or sodium monofluorophosphate is a soluble fluoride salt which provides soluble fluoride in amount of 50 to 25,000 ppm fluoride, such as in an amount of about 1000 ppm-1500 ppm, for example in an amount of about 1450 ppm. In some embodiments the fluoride source is sodium fluoride in an amount about 0.32% by wt, based on the total weight of the composition. In some embodiments, the fluoride source is stannous fluoride. Some embodiments set out above further comprise a preservative selected from: benzyl alcohol, Methylisothizolinone (“MIT”), Sodium bicarbonate, sodium methyl cocoyl taurate (tauranol), lauryl alcohol, and polyphosphate. Some embodiments set out above further comprise benzyl alcohol in an amount of from 0.1-0.8% wt %, or from 0.3-0.5% wt %, or about 0.4 wt % based on the total weight of the composition. In some embodiments, the oral care composition comprises about 1.0% zinc oxide, about 0.5% zinc citrate, about 1.5% L-arginine, about 1450 ppm sodium fluoride, and optionally about benzyl alcohol 0.1 wt. % and/or about 5% small particle silica (e.g., AC43), based on the total weight of the composition. In some embodiments, the oral care composition comprises about 1.0% zinc oxide, about 0.5% zinc citrate, about 5% L-arginine, about 1450 ppm sodium fluoride, and optionally about benzyl alcohol 0.1 wt. % and/or about 5% small particle silica (e.g., AC43), based on the total weight of the composition. In some embodiments set out above, the oral care composition may comprise about 1.0% zinc oxide, about 0.5% zinc citrate, about 1.5% L-arginine, about 0.22%-0.32% sodium fluoride, about 0.5% tetrasodium pyrophosphate, and optionally about benzyl alcohol 0.1 wt. %, based on the total weight of the composition. In some embodiments set out above, the oral care composition may be any of the following oral care compositions selected from the group consisting of: a toothpaste or a dentifrice, a mouthwash or a mouth rinse, a topical oral gel, and a denture cleanser.

### Example 7

Oral compositions that comprise arginine are disclosed in WO 2017/223169, which is incorporated herein by reference. In some embodiments, the oral care composition comprises: arginine in free or salt form, zinc oxide and zinc citrate and a fluoride source comprising stannous fluoride. In some embodiments, the oral care compositions comprise zingerone, zinc oxide, zinc citrate; and a stannous fluoride. In some embodiments, the zingerone is present in an amount of from 0.01% to 1%, based on the total weight of the composition. In some embodiments, the ratio of the amount of zinc oxide (by wt %) to zinc citrate (by wt %) is 2:1, 2.5:1, 3:1, 3.5:1 or 4:1, based on the total weight of the composition. In some embodiments, the zinc citrate is present in an amount of from 0.25 to 1.0 wt % and zinc oxide is present in an amount of from 0.75 to 1.25 wt %, based on the total weight of the composition. In some embodiments, the zinc citrate is present in an amount of about 0.5 wt % and zinc is present in an amount of about 1.0% based on the total weight of the composition. In some embodiments, the stannous fluoride is present in an amount of 0.1 wt, % to 2 wt. %, based on the total weight of the composition. Some embodiments further comprise synthetic amorphous precipitated abrasive silica in an amount of from 1%-25% by wt, based on the total weight of the composition and/or a high cleaning silica in an amount of from 1 wt %-15 wt %, based on the total weight of the composition. Some embodiments further comprise an effective amount of one or more alkali phosphate salts, for example sodium tripolyphosphate in an amount of from 1-5 wt %, based on the total weight of the composition. Some embodiments further comprise citric acid in an amount of from 0.1-3 wt. %, and citrate ion, for example trisodium citrate dihydrate, in an amount of from 0.1-5 wt. %, based on the total weight of the composition. Some embodiments further comprise carboxymethyl cellulose in an amount of from 0.1 wt, %-1.5 wt. %, based on the total weight of the composition. Some embodiments further comprise an anionic surfactant, e.g., sodium lauryl sulfate, in an amount of from 0.5-5% by weight, based on the total weight of the composition. Some embodiments further comprise an amphoteric surfactant in an amount of from 0.5-5%, based on the total weight of the composition. Some embodiments further comprise a PVM/MA copolymer, such as for example a Gantrez polymer, in an amount of from 0.1-5 wt. %, based on the total weight of the composition. Some embodiments further comprise microcrystalline cellulose/sodium carboxymethylcellulose. Some embodiments further comprise one or both of polyethylene glycol in an amount of from 1-6%; and propylene glycol in an amount of from 1-6%, based on the total weight of the composition. Some embodiments further comprise polyvinylpyrrolidone (PVP) in an amount of from 0.5-3 wt. %, based on the total weight of the composition. Some embodiments further comprise from 5%-40% free water by weight, based on the total weight of the composition. Some embodiments further comprise one or more thickening agents, e.g., sodium carboxymethyl cellulose and sodium carboxy methyl hydroxyethyl cellulose. In some embodiments, the oral care composition comprises: about 0.1-0.3% zingerone; about 1.0% zinc oxide; about 0.5% zinc citrate, and about 0.4%-0.5% stannous fluoride. In some embodiments, the oral care composition comprises: about 0.1-0.3% zingerone; about 1.0% zinc oxide; about 0.5% zinc citrate, about 0.4%-0.5% stannous fluoride; and about 1.2% abrasive silica and may, in some such embodiments, further comprise about 7% wt % high cleaning silica, based on the total weight of the composition, and/or a surfactant system comprising one or both of an anionic surfactant in an amount of from 0.5-5%, by weight; and/or an amphoteric surfactant in an amount of from 0.5-5% by weight, based on the total weight of the composition. Some embodiments further comprise sodium tripolyphosphate in an amount of from 1-5 wt %, based on the total weight of the composition and/or sodium phosphate in an amount of from 0.5 wt %-5 wt %, based on the total weight of the composition. In some embodiments, such compositions are dentifrices (e.g., a toothpaste or oral gel), Example 8

Test dentifrices comprising arginine, zinc oxide, zinc citrate and a source of fluoride were prepared as shown in Formulation Tables A-E:

Formulation Table A

| Ingredient | Compound I |
|---|---|
| Humectants | 20.0-25.0 |
| Non-ionic surfactant | 1.0-2.0 |
| Amphoteric surfactant | 3.0-4.0 |
| Flavoring/fragrance/coloring agent | 2.0-3.0 |
| Polymers | 10.0-15.0 |
| pH adjusting agents | 1.5-3.0 |
| Precipitated Calcium Carbonate | 35 |
| Zinc citrate trihydrate | 0.5 |
| Zinc oxide | 1.0 |
| Sodium Fluoride -USP, EP | 0.32 |
| Arginine Bicarbonate | 13.86 |
| Demineralized water | QS |

Formulation Table B

| | Com- | Com- | Com- | Com- |
|---|---|---|---|---|
| Ingredient | pound A | pound B | pound C | pound D |
| Humectants | 25.0-40.0 | 25.0-40.0 | 25.0-40.0 | 25.0-40.0 |
| Anionic surfactant | 1.0-3.0 | 1.0-3.0 | 1.0-3.0 | 1.0-3.0 |
| Flavoring/fragrance/ | 2.5-4.0 | 2.5-4.0 | 2.5-4.0 | 2.5-4.0 |
| coloring agent | | | | |
| Polymers | 4.0-6.0 | 4.0-6.0 | 4.0-6.0 | 4.0-6.0 |
| pH adjusting agents | 5.0-6.0 | 5.0-6.0 | 5.0-6.0 | 5.0-6.0 |
| Synthetic Amorphous | 16.00 | 21.37 | 17.92 | 7.81 |
| Precipitated Silica | | | | |
| Alumina | 0.02 | 0.01 | 0.01 | 0.01 |
| Silica | — | — | — | 15.0 |
| Lauryl alcohol | 0.02 | 0.02 | 0.02 | 0.02 |
| Zinc citrate | 0.5 | 0.5 | 0.5 | 0.5 |
| Zinc oxide | 1.0 | 1.0 | 1.0 | 1.0 |
| Sodium Fluoride - | 0.32 | 0.32 | 0.32 | 0.32 |
| USP, EP | | | | |
| L-Arginine Bicarbonate | 5.0 | 5.0 | 5.0 | 5.0 |
| Demineralized water | QS | QS | QS | QS |

Formulation Table C

| | Com- | Com- | Com- |
|---|---|---|---|
| Ingredient | pound E | pound F | pound G |
| Humectants | 25.0-40.0 | 25.0-40.0 | 25.0-40.0 |
| Anionic surfactant | 1.0-3.0 | 1.0-3.0 | 1.0-3.0 |
| Non-ionic surfactant | 0.1-1.0 | 0.1-1.0 | 0.1-1.0 |
| Amphoteric surfactant | 0.1-1.0 | 0.1-1.0 | 0.1-1.0 |
| Flavoring/fragrance/coloring agent | 4.0-6.0 | 4.0-6.0 | 4.0-6.0 |
| Polymers | 0.1-2.0 | 0.1-2.0 | 0.1-2.0 |
| pH adjusting agents | 5.0-6.0 | 5.0-6.0 | 5.0-6.0 |
| Thickener | 6.0 | 6.5 | 7.0 |
| Alumina | 0.1 | 0.1 | 0.1 |
| Synthetic Amorphous | 17.6 | 8.8 | 22.4 |
| Precipitated Silica | | | |
| Silica | — | 15.0 | — |
| Benzyl alcohol | 0.1 | 0.1 | 0.1 |
| Synthetic Amorphous Silica | 5.0 | 5.0 | 5.0 |
| Zinc citrate | 0.5 | 0.5 | 0.5 |
| Zinc oxide | 1.0 | 1.0 | 1.0 |
| Sodium Fluoride - USP, EP | 0.32 | 0.32 | 0.32 |
| L-Arginine Bicarbonate | 1.5 | 1.5 | 1.5 |
| Demineralized water | QS | QS | QS |

Formulation Table D

| Ingredient | Compound H | Compound I |
|---|---|---|
| Humectants | 45.0-55.0 | 35.0-45.0 |
| Abrasives | 14.0-16.0 | 9.0-11.0 |
| Anionic surfactant | 1.0-3.0 | 1.0-3.0 |
| Non-ionic surfactant | 0.1-1.0 | — |
| Amphoteric surfactant | 1.0-2.0 | — |
| Flavoring/fragrance/coloring agent | 1.0-3.0 | 2.0-4.0 |
| Polymers | 0.1-2.0 | 3.0-8.0 |
| pH adjusting agents | 0.1-2.0 | 4.0-8.0 |
| Silica Thickener | 5.0 | 5.0-10.0 |
| Benzyl alcohol | 0.1 | — |
| Zinc citrate trihydrate | 0.5 | 0.5 |
| Zinc oxide | 1.0 | 1.0 |
| Sodium Fluoride - USP, EP | 0.32 | 0.32 |
| L-Arginine | 1.5 | 5.0 |
| Demineralized water | QS | QS |

Formulation Table E

| | Com- | Com- | Com- |
|---|---|---|---|
| Ingredient | pound J | pound K | pound L |
| Humectants | 20.0-50.0 | 20.0-50.0 | 20.0-50.0 |
| Abrasives | 5.0-20.0 | 5.0-20.0 | 5.0-20.0 |
| Anionic surfactant | 1.0-3.0 | 1.0-3.0 | 1.0-3.0 |
| Non-ionic surfactant | 0.1-1.0 | 0.1-1.0 | 0.1-1.0 |
| Amphoteric surfactant | 0.1-2.0 | 0.1-2.0 | 0.1-2.0 |
| Flavoring/fragrance/coloring agent | 1.0-5.0 | 1.0-5.0 | 1.0-5.0 |
| Polymers | 0.1-2.0 | 0.1-2.0 | 0.1-2.0 |
| pH adjusting agents | 0.1-2.0 | 0.1-2.0 | 0.1-2.0 |
| Thickener | 6.0 | 6.5 | 7.0 |
| Dental type silica | — | — | 15.0 |
| High cleaning silica | — | 15.0 | — |
| Synthetic Abrasives | 10.0 | — | — |
| Synthetic Amorphous Silica | 5.0 | 5.0 | 5.0 |
| Benzyl alcohol | 0.4 | 0.4 | 0.4 |
| Zinc citrate trihydrate | 0.5 | 0.5 | 0.5 |
| Zinc oxide | 1.0 | 1.0 | 1.0 |
| Sodium Fluoride - USP, EP | 0.32 | 0.32 | 0.32 |
| L-Arginine | 1.5 | 1.5 | 1.5 |
| Demineralized water | QS | QS | QS |

### Example 9

Test dentifrices comprising arginine, zinc oxide, zinc citrate and stannous fluoride were prepared as shown in Formulation Table F:

Formulation Table F

Ingredient

| | | | |
|---|---|---|---|
| Humectants | 20.0-60.0 | 20.0-50.0 | 20.0-50.0 |
| Abrasives | 10.0-40.0 | 5.0-20.0 | 5.0-20.0 |
| Anionic surfactant | 1.0-3.0 | 1.0-3.0 | 1.0-3.0 |
| Amphoteric surfactant | 0.5-1.5 | 0.1-2.0 | 0.1-2.0 |
| Flavoring/fragrance/coloring agent | 0.5-5.0 | 1.0-5.0 | 1.0-5.0 |
| Polymers | 1.0-10.0 | 0.1-2.0 | 0.1-2.0 |
| pH adjusting agents | 1.0-10.0 | 0.1-2.0 | 0.1-2.0 |
| Zinc citrate | 0.25-1.0 | 0.5 | 0.5 |
| Zinc oxide | 0.75-1.25 | 1.0 | 1.0 |
| Stannous Fluoride | 0.1-1.0 | 0.32 | 0.32 |
| L-Arginine | 0.1-10.0 | 1.5 | 1.5 |
| Demineralized water | QS | QS | QS |

1. A method of increasing sIgA levels in an individual's oral cavity, the method comprising:
applying at least twice daily for at least seven consecutive days to the individual's oral cavity an oral care composition that comprises an effective amount of zinc oxide, zinc citrate, and arginine to increase sIgA in the individual's oral cavity.

2. The method of claim 1, wherein the oral care composition is applied by brushing with a tooth brush for about 1 minute or more.

3. The method of claim 1, wherein:
the zinc oxide is present in the oral care composition in an amount of from 0.75 to 1.25 wt. %,
the zinc citrate is present in the oral care composition in an amount of from 0.25 to 1 wt. %, and
the arginine is present in the oral care composition in an amount of from 0.1 to 15 wt. %, based on the total weight of the composition, the weight of the basic amino acid being calculated as free form.

4. The method of claim 3 wherein the arginine is L-arginine.

5. The method of claim 4 wherein the arginine is in free form.

6. The method of claim 4 wherein the arginine is in salt form.

7. The method of claim 3, wherein the weight ratio of the amount of zinc oxide (by wt. %) to zinc citrate (by wt. %) is from about 2:1 to about 4:1, based on the total weight of the composition.

8. The method of claim 3, wherein the weight ratio of the amount of zinc oxide (by wt. %) to zinc citrate (by wt. %) is about 2:1, based on the total weight of the composition.

9. The method of claim 1 wherein the oral care composition further comprises fluoride.

10. The method of claim 9 wherein the oral care composition further comprises stannous fluoride.

11. The method of claim 1, wherein the oral care composition is a toothpaste.

12. (canceled)

13. (canceled)

14. The method of claim 3, wherein the ratio of the amount of zinc oxide (by wt. %) to zinc citrate (by wt. %) is about 2:1, about 2.5:1, about 3:1, about 3.5:1, or about 4:1, based on the total weight of the composition.

15. The method of claim 3, wherein the oral care composition further comprises an abrasive selected from silica, orthophosphates, polymetaphosphates, pyrophosphates, calcium phosphates, calcium carbonates, and a combination of two or more thereof.

16. The method of claim 15, wherein the abrasive comprises an acidic silica abrasive.

17. The method of claim 1, wherein the oral care composition further comprises an amphoteric surfactant comprising a betaine surfactant.

18. The method of claim 3, wherein the oral care composition further comprises an anionic surfactant.

19. The method of claim 3, wherein the oral care composition further comprises a PVM/MA copolymer.

20. The method of claim 1, wherein the oral care composition comprises an amount of zinc oxide, zinc citrate, and arginine effective to increase the concentration of sIgA in the individual's mucosa pellicle in the oral cavity.

21. The method of claim 1, wherein the oral care composition comprises an amount of zinc oxide, zinc citrate, and arginine effective to increase phagocytotic activity of phagocytotic cells in the individual's oral cavity.

22. The method of claim 1, wherein the oral care composition comprises an amount of zinc oxide, zinc citrate, and arginine effective to increase levels of β-Defensin 1 in the individual's oral cavity.
